(19) United States
(12) Patent Application Publication
(10) Pub. No.: US 2025/0288957 A1
(43) Pub. Date: Sep. 18, 2025

(54) INTENSIFIED ULTRAFILTRATION/DIAFILTRATION PROCESSES

(71) Applicant: AMGEN INC., Thousand Oaks, CA (US)

(72) Inventor: Andrew John MALONEY, Boston, MA (US)

(73) Assignee: AMGEN INC., Thousand Oaks, CA (US)

(21) Appl. No.: 19/077,391

(22) Filed: Mar. 12, 2025

Related U.S. Application Data
(60) Provisional application No. 63/565,068, filed on Mar. 14, 2024.

(51) Int. Cl. B01D 61/14; B01D 65/02; C07K 1/34

(52) U.S. Cl. CPC B01D 61/146; B01D 65/02; C07K 1/34; B01D 2315/16; B01D 2321/168

(57) ABSTRACT

Disclosed herein are ultrafiltration/diafiltration (UF/DF) methods comprising the use of diafiltration buffer concentrates, as well as UF/DF methods in which a composition comprising a recombinant protein is concentrated by an ultrafiltration step to a recombinant protein concentration above an intended concentration following a subsequent diafiltration step.

### CROSS-REFERENCE TO RELATED APPLICATIONS

This application claims the benefit of priority to U.S. Provisional Patent Application No. 63/565,068, filed Mar. 14, 2024, which is hereby incorporated by reference in its entirety.

### FIELD

The present disclosure provides ultrafiltration/diafiltration (UF/DF) methods comprising the use of diafiltration buffer concentrates, as well as UF/DF methods in which a composition comprising a recombinant protein is concentrated by an ultrafiltration step to a recombinant protein concentration above an intended concentration following a subsequent diafiltration step.

### BACKGROUND

Ultrafiltration and diafiltration (UF/DF) processes are commonly used in biomanufacturing to formulate a drug substance prior to bulk storage or drug product fill/finish. During a UF/DF process, a recombinant protein-containing composition that has undergone one or more downstream purification processes can be concentrated and buffer-exchanged into a desired formulation matrix. For example, an incoming product pool may be concentrated by ultrafiltration to a target concentration using a semi-permeable membrane filter, buffer-exchanged via diafiltration, optionally further concentrated, and then recovered to obtain a UF/DF pool. In a typical UF/DF operation, buffer exchange and recovery are both performed using the same diafiltration buffer.

These buffer-exchange and final formulation steps can be time and resource intensive, particularly when used as part of an intensified biomanufacturing process with a high associated titer. Specifically, the UF/DF process can be demanding in terms of diafiltration buffer consumption and associated buffer batching requirements due to the number of diavolumes needed during diafiltration (often up to 7 to 10) for near-complete buffer exchange.

Accordingly, there is a need in the art for intensified ultrafiltration/diafiltration methods that maintain process performance while reducing resource requirements, including diafiltration buffer batching requirements.

### SUMMARY

One aspect of the present disclosure provides a method for preparing a formulation comprising a recombinant protein, the method comprising diafiltering a first composition comprising a recombinant protein with a diafiltration buffer to obtain a second composition, wherein the diafiltration buffer is prepared by diluting a diafiltration buffer concentrate.

In some embodiments, the diafiltration buffer is prepared by diluting a diafiltration buffer concentrate with water (e.g., deionized water). In some embodiments, the diafiltration buffer is prepared by diluting a diafiltration buffer concentrate at an intended dilution ratio of 1:1. In some embodiments, the diafiltration buffer is prepared by diluting a diafiltration buffer concentrate at an intended dilution ratio of 1:1 with water (e.g., deionized water).

In some embodiments, the diluting is performed using an in-line dilution system. In some embodiments, the diluting is continuously performed using an in-line dilution system. In some embodiments, a pump of the in-line dilution system has a relative dilution accuracy in the range of 90% to 110% of an intended dilution ratio.

In some embodiments, no other step of the method (e.g., the UF/DF unit operation) uses a diafiltration buffer prepared by dilution from a diafiltration buffer concentrate.

Another aspect of the present disclosure provides a method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer to obtain a second composition,
wherein the first diafiltration buffer is prepared by diluting a diafiltration buffer concentrate.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the diluting is performed using an in-line dilution system. In some embodiments, the diluting is continuously performed using an in-line dilution system. In some embodiments, a pump of the in-line dilution system has a relative dilution accuracy in the range of 90% to 110% of an intended dilution ratio.

In some embodiments, no other step of the method (e.g., the UF/DF unit operation, including, e.g., the recovery process) uses a diafiltration buffer prepared by dilution from a diafiltration buffer concentrate.

Still another aspect of the present disclosure provides a method for preparing a formulation comprising a recombinant protein, the method comprising diafiltering a first composition comprising a recombinant protein with a first diafiltration buffer and a second diafiltration buffer to obtain a second composition, wherein the first diafiltration buffer is prepared by diluting a diafiltration buffer concentrate; and the second diafiltration buffer is not prepared by diluting a diafiltration buffer concentrate.

In some embodiments, the second diafiltration buffer is batch prepared at its intended composition.

In some embodiments, the first diafiltration buffer and the second diafiltration buffer have substantially the same composition.

In some embodiments, the first diafiltration buffer and the second diafiltration buffer contain the same excipients, and each excipient in the first diafiltration buffer is present in the second diafiltration buffer in an amount that is in the range of 90% to 110% of its amount in the second diafiltration buffer.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the diluting is performed using an in-line dilution system. In some embodiments, the diluting is continuously performed using an in-line dilution system. In some embodiments, a pump of the in-line dilution system has a relative dilution accuracy in the range of 90% to 110% of an intended dilution ratio.

In some embodiments, no other step of the method uses a diafiltration buffer prepared by dilution from a diafiltration buffer concentrate.

Another aspect of the present disclosure provides a method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer and a second diafiltration buffer to obtain a second composition,
wherein:
   the first diafiltration buffer is prepared by diluting a diafiltration buffer concentrate;
   the second diafiltration buffer is not prepared by diluting a diafiltration buffer concentrate; and
   the first diafiltration buffer and the second diafiltration buffer have substantially the same composition.

In some embodiments, the second diafiltration buffer is batch prepared at its intended composition.

In some embodiments, the first diafiltration buffer and the second diafiltration buffer contain the same excipients, and each excipient in the first diafiltration buffer is present in the second diafiltration buffer in an amount that is in the range of 90% to 110% of its amount in the second diafiltration buffer.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the diluting is performed using an in-line dilution system. In some embodiments, the diluting is continuously performed using an in-line dilution system. In some embodiments, a pump of the in-line dilution system has a relative dilution accuracy in the range of 90% to 110% of an intended dilution ratio.

In some embodiments, no other step of the method (e.g., the UF/DF unit operation, including, e.g., the recovery process) uses a diafiltration buffer prepared by dilution from a diafiltration buffer concentrate.

Yet another aspect of the disclosure provides a method for preparing a formulation comprising a recombinant protein, the method comprising diafiltering a first composition comprising a recombinant protein with a first diafiltration buffer and a second diafiltration buffer to obtain a second composition, wherein the first diafiltration buffer is a concentrate of the second diafiltration buffer.

In some embodiments, the first diafiltration buffer is at least a 2× concentrate of the second diafiltration buffer. In some embodiments, the first diafiltration buffer is a 2× concentrate of the second diafiltration buffer.

In some embodiments, the method comprises diafiltering with the first diafiltration buffer for 0.5 diavolumes followed by diafiltering with the second diafiltration buffer for 6.5 to 9.5 diavolumes. In some embodiments, the method comprises diafiltering with the first diafiltration buffer for one diavolume followed by diafiltering with the second diafiltration buffer for 6 to 9 diavolumes. In some embodiments, the method comprises diafiltering with the first diafiltration buffer for two diavolumes followed by diafiltering with the second diafiltration buffer for 5 to 8 diavolumes. In some embodiments, the method comprises diafiltering with the first diafiltration buffer for three diavolumes followed by diafiltering with the second diafiltration buffer for 4 to 7 diavolumes.

Another aspect of the disclosure provides a method for preparing a formulation comprising a recombinant protein, the method comprising:
concentrating a first composition comprising a recombinant protein by ultrafiltration to obtain a first concentrated composition; and
diafiltering the first concentrated composition to obtain a second composition, wherein the recombinant protein is present in the first concentrated composition at a concentration that is at least 120% of a concentration of the recombinant protein in the second composition.

In some embodiments, prior to the diafiltering, the first concentrated composition is diluted with a diafiltration buffer. In some embodiments, prior to the diafiltering, the first concentrated composition is diluted with a diafiltration buffer such that the concentration of the recombinant protein in the composition post-dilution is substantially the same as the concentration in the second composition.

Still another aspect of the disclosure provides a method for preparing a formulation comprising a recombinant protein, the method comprising:
concentrating a first composition comprising a recombinant protein by ultrafiltration to obtain a first concentrated composition; and
diafiltering the first concentrated composition with a first diafiltration buffer and a second diafiltration buffer to obtain a second composition, wherein:
the first diafiltration buffer is a concentrate of the second diafiltration buffer; and
the recombinant protein is present in the first concentrated composition at a concentration that is at least 120% of a concentration of the recombinant protein in the second composition.

In some embodiments, prior to the diafiltering, the first concentrated composition is diluted with a diafiltration buffer (e.g., the second diafiltration buffer). In some embodiments, prior to the diafiltering, the first concentrated composition is diluted with a diafiltration buffer (e.g., the second diafiltration buffer) such that the concentration of the recombinant protein in the composition post-dilution is substantially the same as the concentration in the second composition.

In some embodiments, the first diafiltration buffer is at least a 2× concentrate of the second diafiltration buffer. In some embodiments, the first diafiltration buffer is a 2× concentrate of the second diafiltration buffer.

In some embodiments, the method comprises diafiltering with the first diafiltration buffer for 0.5 diavolumes followed by diafiltering with the second diafiltration buffer for 6.5 to 9.5 diavolumes. In some embodiments, the method comprises diafiltering with the first diafiltration buffer for one diavolume followed by diafiltering with the second diafiltration buffer for 6 to 9 diavolumes. In some embodiments, the method comprises diafiltering with the first diafiltration buffer for two diavolumes followed by diafiltering with the second diafiltration buffer for 5 to 8 diavolumes. In some embodiments, the method comprises diafiltering with the first diafiltration buffer for three diavolumes followed by diafiltering with the second diafiltration buffer for 4 to 7 diavolumes.

### BRIEF DESCRIPTION OF THE DRAWINGS

FIG. 1 provides a schematic representation of the batching requirements associated with various diafiltration buffer concentrate amounts (1×-10×).

FIG. 2 provides a schematic representation of the relative delivery error associated with a 2% or a 3% pump error rate at various diafiltration buffer concentrate amounts (1×-10×).

FIG. 3 shows relative changes in predicted UF/DF pool osmolality for intensified UF/DF process scenarios described in Example 1 as compared to a non-intensified process in which a 1× diafiltration buffer is used for the entire UF/DF process.

FIG. 4 shows the process capability (Ppk) relative to product specification limits for osmolality for the simulated scenarios described in Example 1, assuming a worst-case uniform distribution for the pump variability.

FIG. 5 shows the simulated concentration of an excipient under different target ultrafiltration concentration/pre-diafiltration dilution scenarios described in Example 2.

FIG. 6 shows the simulated concentration of an excipient under different expedited buffer exchange scenarios described in Example 2.

### DETAILED DESCRIPTION

Disclosed herein are ultrafiltration/diafiltration (UF/DF) methods comprising the use of diafiltration buffer concentrates. Also disclosed herein are UF/DF methods in which a composition comprising a recombinant protein is concentrated by an ultrafiltration step to a recombinant protein concentration above an intended concentration following a subsequent diafiltration step.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

In some embodiments, “about,” when used in connection with a measurable numerical variable, refers to the indicated value of the variable and to all values of the variable that are within the experimental error of the indicated value (e.g., within the 95% confidence interval for the mean) or ±10% of the indicated value, whichever is greater. In some embodiments, numeric ranges are inclusive of the numbers defining the range (i.e., the endpoints). A composition can be “substantially the same” as a reference composition when the compositions contain the same components, wherein the amount of each composition is within ±10% (e.g., ±5%; ±2.5%) of the amount in the other composition (as determined by the amount in the composition containing a higher amount of the component).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “acid precipitation” refers to a harvest operation in which cell culture pH is reduced to induce precipitation of one or more cell culture impurities.

As used herein, the term “affinity chromatography” (also referred to as “capture chromatography”) refers to a chromatography operation in which a biomolecule (e.g., a recombinant protein) is separated from a mixture based on a selective interaction between the biomolecule and another substance (i.e., a ligand). Affinity chromatography is commonly used in biomanufacturing processes to isolate and concentrate desired recombinant proteins from harvested cell culture fluid. In a typical affinity chromatography operation, a biomolecule in a moving phase selectively binds to or otherwise interacts with a stationary phase while the rest of the moving phase passes through the chromatography material. The biomolecule is then eluted from the stationary phase by changing the conditions in a manner that reduces the affinity between the ligand and the biomolecule. Non-limiting examples of affinity chromatography materials include Protein A, Protein G, Protein A/G, and Protein L materials. Additionally, immobilized metal affinity chromatography (IMAC) can be used to capture proteins that have or have been engineered to have affinity for metal ions.

In some embodiments, protein A affinity chromatography may be employed to capture a recombinant protein of interest. Protein A ligands are highly selective for a wide range of proteins containing an antibody Fc region and provide robust removal of process-related impurities with high target protein yields. Commercially available protein A materials include, but are not limited to, MABSELECT™ SURE Protein A, Protein A Sepharose FAST FLOW™, MABSELECT™ PrismA (Cytiva, Marborough, MA), PROSEP-A™ (Merck Millipore, U.K), TOYOPEARL™ HC-650F Protein A (TosoHass Co., Philadelphia, PA), and AP Plus, Purolite, King of Prussia, PA).

As used herein, the term “antigen-binding protein” refers to a protein or polypeptide that comprises an antigen-binding region or antigen-binding portion that has affinity for another molecule to which it binds (antigen). Antigen-binding proteins include, but are not limited to, antibodies, fusion proteins, VH, VHH, VL, (s)dAb, Fv, light chain (VL-CL), Fd (VH-CH1), heavy chain, Fab, Fab′, F(ab′)2 or “r IgG” (“half antibody” consisting of a heavy chain and a light chain) or a modified antigen-binding portion of a full-length antibody, such as, e.g., a triple-chain antibody-like molecule, a heavy chain only antibody, single-chain variable fragment (scFv), di-scFv or bi(s) scFv, scFv-Fe, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH, or VL, that specifically binds to an antigen or target independently of other variable regions or domains.

As used herein, the term “antibody” generally refers to a tetrameric immunoglobulin protein comprising two light chain polypeptides (about 25 kDa each) and two heavy chain polypeptides (about 50-70 kDa each).

As used herein, the term “light chain” or “immunoglobulin light chain” refers to a polypeptide comprising, from amino terminus (N-terminus) to carboxyl terminus (C-terminus), a single immunoglobulin light chain variable region (VL) and a single immunoglobulin light chain constant domain (CL). The immunoglobulin light chain constant domain (CL) can be a human kappa (κ) or human lambda (λ) constant domain.

As used herein, the term “heavy chain” or “immunoglobulin heavy chain” refers to a polypeptide comprising, from amino terminus (N-terminus) to carboxyl terminus (C-terminus), a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

Papain digestion of antibodies produces two identical antigen-binding proteins, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as Clq binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

As used herein, the term “heavy chain-only antibody” refers to an immunoglobulin protein consisting of two heavy chain polypeptides (such as, e.g., heavy chain polypeptides that are about 50-70 kDa each). A “heavy chain-only antibody” lacks the two light chain polypeptides found in a conventional antibody. Heavy-chain antibodies constitute about one-fourth of the IgG antibodies produced by the camelids, e.g., camels and llamas (Hamers-Casterman C., et al. Nature. 363, 446-448 (1993)). These molecules are formed by two heavy chains but are devoid of light chains. As a consequence, the variable antigen binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 3746 (1999)), and the VHH portion can be readily cloned and expressed in yeast (Frenken, L. G. J., et al. J. Biotechnol. 78, 11-21 (2000)). Their levels of expression, solubility, and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy chain-only antibody” is a dimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, the term “three-chain antibody like molecule” or “TCA” refers to an antibody-like molecule comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

As used herein, the term “cell culture” or “culture” refers to the growth and propagation of cells outside of a multicellular organism or tissue. Suitable culture conditions for mammalian and bacterial cells are known in the art. (See, e.g., Animal cell culture: A Practical Approach, D. Rickwood, ed., Oxford University Press, New York (1992).) Mammalian cells may be cultured in suspension or while attached to a solid substrate. In some embodiments, fluidized bed bioreactors, hollow fiber bioreactors, roller bottles, shake flasks, and/or stirred tank bioreactors, with or without microcarriers, may be used for cell culture. In some embodiments, 500 L to 2000 L bioreactors are used for cell culture (e.g., as part of a seed train). In some embodiments, 1000 L to 2000 L bioreactors are used for cell culture (e.g., as part of a seed train).

As used herein, the term “connected,” in reference to unit operations, refers to a direct connection or mechanism that allows for continuous flow between one or more unit operations in a single operational cycle.

As used herein, the term “continuous,” in reference to unit operations, refers to a direct connection or mechanism that allows for continuous flow between one or more unit operations.

As used herein, the term “dynamic binding capacity,” in reference to a chromatography material, refers to the amount of product, e.g. polypeptide, the material will bind under actual flow conditions before significant breakthrough of unbound product occurs.

As used herein, the term “expression vector” or “expression construct” refers to a recombinant DNA molecule containing a desired coding sequence and appropriate nucleic acid control sequences necessary for the expression of the operably linked coding sequence in a particular host cell, e.g., a mammalian host cell. Vectors can include viral vectors, non-episomal mammalian vectors, plasmids, and other non-viral vectors. An expression vector can include sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

As used herein, a “fusion protein” is a protein that contains at least one polypeptide fused or linked to a heterologous polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell to produce the fusion protein. The fusion protein may comprise a fragment from an immunoglobulin protein, such as an Fc region, fused or linked to a ligand polypeptide, a receptor polypeptide, a hormone, cytokine, growth factor, an enzyme, or other polypeptide that is not a component of an immunoglobulin.

As used herein, the term “polypeptide” refers to a polymer of amino acids comprising at least 50 amino acids, such as, e.g., at least 100 amino acids.

As used herein, the term “polishing chromatography” refers to a chromatography operation performed after a capture or affinity chromatography operation to remove remaining impurities and obtain a more highly purified composition and/or recombinant protein. Common impurities removed during polishing steps include, but are not limited to, product-related impurities (e.g., HMW and LMW species), host cell proteins, DNA, leached protein A, viral contaminants, and endotoxins. In addition, typical chromatography techniques used for polishing include, but are not limited to, ion exchange chromatography (IEX), hydrophobic interaction chromatography (HIC), and multimodal (or mixed mode) chromatography (MMC).

“Anion exchange chromatography” (AEX) refers to a form of ion exchange chromatography performed on a solid phase medium (e.g., resin or membrane) that is positively charged and has the capacity to exchange anions with anions in an aqueous solution passed over or through the solid phase. AEX chromatography is used, for example, for viral clearance and impurity removal. Commercially available anion exchange media include, but are not limited to, sulphopropyl (SP) immobilized on agarose (e.g., Source 15 Q, Capto™ Q, Q-SEPHAROSE FAST FLOW™ (Cytiva), FRACTOGEL TMAE™, FRACTOGEL EDM DEAE™, (EMD Merck), TOYOPEARL Super Q® and TOYOPEARL NH2-750F (Tosoh Bioscience), POROS HQ™, and POROS XQ™ (ThermoFisher).

“Cation exchange chromatography” (CEX) refers to a form of ion exchange chromatography performed on a solid phase medium (e.g., resin or membrane) that is negatively charged and has the capacity to exchange cations with cations in an aqueous solution passed over or through the solid phase. The charge may be provided by attaching one or more charged ligands to the solid phase, e.g., via covalent linkage. Alternatively or additionally, the charge may be an inherent property of the solid phase (e.g., silica, which has an overall negative charge). CEX chromatography is typically used to remove high molecular weight (HMW) contaminants, process related impurities, and/or viral contaminants. Commercially available cation exchange media include, but are not limited to, sulphopropyl (SP) immobilized on agarose (e.g., SPSEPHAROSE FAST FLOW™, SP SEPHAROSE FAST FLOW XL™ or SP-SEPHAROSE HIGH PERFORMANCE™, CAPTO S™ CAPTO SP ImpRes™, CAPTO S ImpAct™ (Cytiva), FRACTOGEL-SO3™, FRACTOGEL-SE HICAP™, and FRACTOPREP™ (EMD Merck, Darmstadt, Germany), TOYOPEARL® XS, TOYOPEARL® HS (Tosoh Bioscience, King of Prussia, PA), UNOsphere™ (BioRad, Hercules, CA), S Ceramic Hyper™ DF (Pall, Port Washington, NY), POROS™ (ThermoFisher, Waltham, MA), ESHMUNO® CSP and ESHMUNO® CP-FT (Millipore Sigma, Darmstadt, Germany).

“Hydrophobic interaction chromatography” (HIC) refers to chromatography performed on a solid phase medium that makes use of the interaction between hydrophobic ligands and hydrophobic residues on the surface of a solute. Commercially available hydrophobic interaction chromatography media include, but are not limited to, Phenyl Sephrose™ (Cytiva), TOYOPEARL Hexyl (Tosoh Bioscience), and Capto™ Phenyl (Cytiva).

“Mixed-mode or multi-modal chromatography” (MMC) refers to chromatography that makes use of more than one form of interaction between the stationary phase and analyte to achieve separation. MMC differs from single mode chromatography in that two or more interaction types, such as, e.g., electrostatic, hydrogen bonding, and/or hydrophobic interactions, contribute significantly to the retention of solutes. Commercially available multi-modal chromatography media include, but are not limited to, Capto™ Adhere, Capto™ MMC Impress, Capto MMC, (Cytiva), PPA Hypercel, MEP Hypercell, HEA Hypercell (Pall Corporation, Port Washington, NY). Eshmuno™ HCX (Merk Millipore), and Toyopearl™ MX-Trp-650M (Tosoh Bioscience).

Polishing chromatography unit operations make use of materials (e.g., resins and/or membranes) containing agents that can be operated in a variety of modes, two of which are bind-and-elute mode and flow-though mode. In bind-and-elute chromatography, a biomolecule of interest is usually loaded onto the chromatography material to maximize dynamic binding capacity and then wash and elution conditions are utilized to maximize product purity in the eluate. By contrast, in flow-through chromatography, load conditions are employed that allow impurities to bind to the chromatography material while the biomolecule of interest passes through. Relative to bind-and-elute chromatography, flow-through chromatography allows for higher load densities for many biomolecules.

In addition to the two most common modes, weak partitioning chromatography, overload chromatography, and frontal chromatography modes may also be employed in purification processes. In weak partitioning chromatography, an isocratic separation method, flow-through mode is altered by identifying solution conditions that promote weak binding of a biomolecule to resin, in addition to binding of one or more impurities, with a low product partition coefficient in the range of 0.1-20. In overload chromatography, the biomolecule of interest is loaded onto the chromatography material beyond the dynamic binding capacity of the material. Additionally, frontal chromatography mode allows for a continuous, high-density feed (containing the protein of interest and at least one impurity) onto the chromatography medium. In frontal chromatography, the separation of the protein of interest from impurities and contaminants is driven by the binding affinity of the components in the load feed for the chromatography medium. The amount of the protein of interest that may be loaded on and bound to the chromatography medium in frontal mode is typically dependent on the amount of more highly charged impurities/contaminants, such as product-related impurities, in the load feed. Initially, all the components in the load feed will bind to the chromatography medium. Separation of the product of interest from the impurities/contaminants is driven by affinity for the chromatography medium. When the chromatography medium reaches saturation binding, those components in the load feed having a greater affinity for the chromatography medium (typically product-related impurities such as a HMW species) will displace proteins having a weaker affinity (e.g., the product of interest) resulting in the separation of the proteins with weaker affinity from the chromatography medium. These proteins exit the column in the load flow-through. As loading proceeds the bound proteins are continuously displaced in order of increasing affinity for the chromatography medium until the column is at or near saturation with proteins having greater affinity than the protein of interest.

As used herein, the term “partition coefficient” or “product partition coefficient” (Kr) refers to the molar concentration of product, e.g., recombinant protein, bound to the stationary phase divided by the molar concentration of the product in the mobile phase during a chromatography step.

As used herein, the term “purified,” when used in relation to a composition, refers to a composition wherein at least one impurity is present at a lower concentration in the purified composition relative to the composition as it existed prior to one or more unit operations. Additionally, a “purified” recombinant protein (e.g., a purified antibody) refers to a recombinant purity which has been increased in purity, such that it exists in a form that is more pure than it exists in its natural environment and/or when initially synthesized and/or amplified under laboratory conditions. Purity is a relative term and does not necessarily refer to absolute purity.

As used herein, the term “recombinant protein” refers to a heterologous protein produced by a host cell transfected with a nucleic acid encoding the protein when the host cell is cultivated in cell culture.

As used herein, the term “unit operation” refers to a functional step that is performed as part of a process of purifying a recombinant protein of interest. Unit operations can be designed to achieve a single objective or multiple objectives, such as capture and virus inactivating steps. Unit operations can also include holding or storing steps between processing steps.

As used herein, the term “virus prefilter” refers to a filter upstream of and in fluid communication with a virus filter, which is capable of binding one or more bioprocess impurities to increase the flux and/or throughput of the virus filter.

As used herein, the term “concentrate” refers to a composition that contains the same components as a reference composition, wherein each component is present at a higher concentration as the same component in the reference composition, and further wherein the ratio of the amount of each component in the concentrate to the amount in the reference composition is substantially the same. In some embodiments, a concentrate may be prepared by concentrating a reference composition. In other embodiments, a concentrate may be prepared by batching, e.g., without a concentrating step by adding additional amounts of each component to a solution (e.g., twice the amount as for a reference composition for a 2× concentrate) relative to what would be added during the preparation of a reference composition.

As used herein, the terms “ultrafiltering,” “ultrafiltration,” and “UF” refer to the use of a semi-permeable membrane to separate different molecules or concentrate like molecules. In some embodiments, active force is applied to drive the filtration process. Many types of semi-permeable membranes are commercially available and may be suitable for use in certain UF process, including, but not limited to, regenerated cellulose, Pellicon™ (MilliporeSigma, Danvers, MA), stabilized cellulose, Sartocon® Slice, Sartocon® ECO Hydrosart® (Sartorius, Goettingen, Germany), and polyethersulfone (PES) membrane, Omega (Pall Corporation, Port Washington, NY).

As used herein, the terms “diafiltering,” “diafiltration,” and “DF” refer to the use of a semi-permeable membrane to remove, replace, or lower the concentration of certain components (e.g., salts) in a solution or mixture. In some embodiments, diafiltration enables buffer exchange by adding new buffer to the retentate (i.e., the composition retained on the retentate side of the membrane containing molecules too large to pass through the membrane) from an ultrafiltration process.

As used herein, the term “diavolume” is defined as the “total buffer volume introduced to the operation during diafiltration [divided] by the [initial] retentate volume” (i.e., the number of volumetric equivalents to the initial retentate volume used during diafiltration).

### Non-Limiting Example Features

Without limitation, some example embodiments/features of the present disclosure include:

E1. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer to obtain a second composition,
wherein the first diafiltration buffer is prepared by diluting a diafiltration buffer concentrate.
   E2. The method of E1, wherein the diafiltering comprises diafiltering the first concentrated composition with 6 to 10 (such as, e.g., 7 to 10; 6 to 9; 6; 7; 8, 9; 10) diavolumes of the first diafiltration buffer.
   E3. The method of E1 or E2, wherein the diluting is performed using an in-line dilution system.
   E4. The method of any one of E1-E3, wherein the diluting is continuously performed using an in-line dilution system.
   E5. The method of E3 or E4, wherein a pump of the in-line dilution system has a relative dilution accuracy in the range of 90% to 110% of an intended dilution ratio.
   E6. The method of any one of E1-E5, wherein the first diafiltration buffer is prepared by diluting a diafiltration buffer concentrate with water (e.g., deionized water).
   E7. The method of any one of E1-E6, wherein the first diafiltration buffer is prepared by diluting a diafiltration buffer concentrate at an intended dilution ratio of 1:1.
   E8. The method of any one of E1-E7, wherein the first diafiltration buffer is prepared by diluting a diafiltration buffer concentrate at an intended dilution ratio of 1:1 with water (e.g., deionized water).
   E9. The method of any one of E1-E8, wherein the first diafiltration buffer is prepared by diluting a diafiltration buffer concentrate at an intended dilution ratio of 1:1 with water (e.g., deionized water) using an in-line dilution system.
   E10. The method of any one of E1-E9, wherein the first diafiltration buffer is continuously prepared by diluting a diafiltration buffer concentrate at an intended dilution ratio of 1:1 with water (e.g., deionized water) using an in-line dilution system.
   E11. The method of any one of E1-E10, wherein the first diafiltration buffer is continuously prepared by diluting a diafiltration buffer concentrate at an intended dilution ratio of 1:1 with water (e.g., deionized water) using an in-line dilution system, wherein a pump of the in-line dilution system has a relative dilution accuracy in the range of 90% to 110% of an intended dilution ratio.
   E12. The method of any one of E1-E11, wherein the recombinant protein is present in the first concentrated composition at a concentration that is at least 100% of a concentration of the recombinant protein in the second composition and the first concentrated composition is optionally diluted with a diafiltration buffer prior to the diafiltering, optionally wherein the first concentrated composition is diluted such that the concentration of the recombinant protein in the composition post-dilution is substantially the same as the concentration in the second composition.
   E13. The method of any one of E1-E12, wherein the recombinant protein is present in the first concentrated composition at a concentration that is at least 120% of a concentration of the recombinant protein in the second composition and the first concentrated composition is optionally diluted with a diafiltration buffer prior to the diafiltering, optionally wherein the first concentrated composition is diluted such that the concentration of the recombinant protein in the composition post-dilution is substantially the same as the concentration in the second composition.
   E14. The method of any one of E1-E13, wherein the recombinant protein is present in the first concentrated composition at a concentration that is at least 140% of a concentration of the recombinant protein in the second composition and the first concentrated composition is optionally diluted with a diafiltration buffer prior to the diafiltering, optionally wherein the first concentrated composition is diluted such that the concentration of the recombinant protein in the composition post-dilution is substantially the same as the concentration in the second composition.
   E15. The method of any one of E1-E14, wherein the recombinant protein is present in the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering, optionally wherein the first concentrated composition is diluted such that the concentration of the recombinant protein in the composition post-dilution is substantially the same as the concentration in the second composition.
   E16. The method of any one of E1-15, wherein no other step of the method uses a diafiltration buffer prepared by dilution from a diafiltration buffer concentrate.
   E17. The method of any one of E1-E16, wherein the method further comprises:
concentrating the second composition to obtain a second concentrated composition;
flushing the equilibrated UF/DF membrane filter with a second diafiltration buffer to obtain a recovery composition, wherein the second diafiltration buffer is not prepared by diluting a diafiltration buffer concentrate; and
combining the recovery composition and the second concentrated composition to obtain a third composition.
   E18. The method of E17, wherein concentrating the second composition comprises ultrafiltering using the equilibrated UF/DF membrane filter.
   E19. The method of any one of E1-E16, wherein the method further comprises:
flushing the equilibrated UF/DF membrane filter with a second diafiltration buffer to obtain a recovery composition, wherein the second diafiltration buffer is not prepared by diluting a diafiltration buffer concentrate; and
combining the recovery composition and the second composition to obtain a third composition.
   E20. The method of any one of E17-E19, wherein the first diafiltration buffer and the second diafiltration buffer contain the same excipients, and each excipient in the first diafiltration buffer is present in the second diafiltration buffer in an amount that is in the range of 90% to 110% of its amount in the second diafiltration buffer.
   E21. The method of any one of E17-E20, wherein the first diafiltration buffer and the second diafiltration buffer have substantially the same composition.
   E22. The method of any one of E17-E21, wherein the second diafiltration buffer is batch prepared at its intended composition.
   E23. A method for preparing a formulation comprising a recombinant protein, the method comprising diafiltering a first composition comprising a recombinant protein with a first diafiltration buffer and a second diafiltration buffer to obtain a second composition, wherein the first diafiltration buffer is prepared by diluting a diafiltration buffer concentrate, and the second diafiltration buffer is not prepared by diluting a diafiltration buffer concentrate.
   E24. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer and a second diafiltration buffer to obtain a second composition,
wherein:
   the first diafiltration buffer is prepared by diluting a diafiltration buffer concentrate; and
   the second diafiltration buffer is not prepared by diluting a diafiltration buffer concentrate.
      E25. The method of E23 or E24, wherein the first diafiltration buffer and the second diafiltration buffer contain the same excipients, and each excipient in the first diafiltration buffer is present in the second diafiltration buffer in an amount that is in the range of 90% to 110% of its amount in the second diafiltration buffer.
      E26. The method of any one of E23-E25, wherein the first diafiltration buffer and the second diafiltration buffer have substantially the same composition.
      E27. The method of any one of E23-E21, wherein the second diafiltration buffer is batch prepared at its intended composition.
      E28. The method of any one of E23-E27, wherein the diafiltering comprises diafiltering with the first diafiltration buffer for 6 to 6.5 diavolumes followed by diafiltering with the second diafiltration buffer for 0.5 to 1 diavolumes.
      E29. The method of any one of E23-E28, wherein the diafiltering comprises diafiltering with the first diafiltration buffer for 6 to 6.5 diavolumes followed by diafiltering with the second diafiltration buffer for 0.5 to 1 diavolumes, wherein the total number of diavolumes used during the diafiltering is 7.
      E30. The method of any one of E23-E29, wherein the diafiltering comprises diafiltering with the first diafiltration buffer for 6.5 diavolumes followed by diafiltering with the second diafiltration buffer for 0.5 diavolumes.
      E31. The method of any one of E23-E29, wherein the diafiltering comprises diafiltering with the first diafiltration buffer for 6 diavolumes followed by diafiltering with the second diafiltration buffer for 1 diavolume.
      E32. The method of any one of E23-E31, wherein the diluting is performed using an in-line dilution system.
      E33. The method of any one of E23-E32, wherein the diluting is continuously performed using an in-line dilution system.
      E34. The method of E32 or E33, wherein a pump of the in-line dilution system has a relative dilution accuracy in the range of 90% to 110% of an intended dilution ratio.
      E35. The method of any one of E23-E34, wherein the first diafiltration buffer is prepared by diluting a diafiltration buffer concentrate with water (e.g., deionized water).
      E36. The method of any one of E23-E35, wherein the first diafiltration buffer is prepared by diluting a diafiltration buffer concentrate at an intended dilution ratio of 1:1.
      E37. The method of any one of E23-E36, wherein the first diafiltration buffer is prepared by diluting a diafiltration buffer concentrate at an intended dilution ratio of 1:1 with water (e.g., deionized water).
      E38. The method of any one of E23-E37, wherein the first diafiltration buffer is prepared by diluting a diafiltration buffer concentrate at an intended dilution ratio of 1:1 with water (e.g., deionized water) using an in-line dilution system.
      E39. The method of any one of E23-E38, wherein the first diafiltration buffer is continuously prepared by diluting a diafiltration buffer concentrate at an intended dilution ratio of 1:1 with water (e.g., deionized water) using an in-line dilution system.
      E40. The method of any one of E23-E39, wherein the first diafiltration buffer is continuously prepared by diluting a diafiltration buffer concentrate at an intended dilution ratio of 1:1 with water (e.g., deionized water) using an in-line dilution system, wherein a pump of the in-line dilution system has a relative dilution accuracy in the range of 90% to 110% of an intended dilution ratio.
      E41. The method of any one of E23-E40, wherein, prior to the diafiltering, the recombinant protein is present in the first composition or the first concentrated composition at a concentration that is at least 100% of a concentration of the recombinant protein in the second composition and the first composition or the first concentrated composition is optionally diluted with a diafiltration buffer prior to the diafiltering, optionally wherein the first composition or the first concentrated composition is diluted such that the concentration of the recombinant protein in the composition post-dilution is substantially the same as the concentration in the second composition.
      E42. The method of any one of E23-E41, wherein, prior to the diafiltering, the recombinant protein is present in the first composition or the first concentrated composition at a concentration that is at least 120% of a concentration of the recombinant protein in the second composition and the first composition or the first concentrated composition is optionally diluted with a diafiltration buffer prior to the diafiltering, optionally wherein the first composition or the first concentrated composition is diluted such that the concentration of the recombinant protein in the composition post-dilution is substantially the same as the concentration in the second composition.
      E43. The method of any one of E23-E42, wherein, prior to the diafiltering, the recombinant protein is present in the first composition or the first concentrated composition at a concentration that is at least 140% of a concentration of the recombinant protein in the second composition and the first composition or the first concentrated composition is optionally diluted with a diafiltration buffer prior to the diafiltering, optionally wherein the first composition or the first concentrated composition is diluted such that the concentration of the recombinant protein in the composition post-dilution is substantially the same as the concentration in the second composition.
      E44. The method of any one of E23-E41, wherein, prior to the diafiltering, the recombinant protein is present in the first composition or the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first composition or the first concentrated composition is optionally diluted with a diafiltration buffer prior to the diafiltering, optionally wherein the first composition or the first concentrated composition is diluted such that the concentration of the recombinant protein in the composition post-dilution is substantially the same as the concentration in the second composition.
      E45. The method of any one of E23-E44, wherein no other step of the method uses a diafiltration buffer prepared by dilution from a diafiltration buffer concentrate.
      E46. The method of any one of E23-E45, wherein the method further comprises:
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second composition to obtain a third composition.
   E47. The method of any one of E23-E45, wherein the method further comprises:
concentrating the second composition to obtain a second concentrated composition;
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second concentrated composition to obtain a third composition.
   E48. The method of E47, wherein concentrating the second composition comprises ultrafiltering using the equilibrated UF/DF membrane filter.
   E49. The method of any one of E46-E48, wherein the first diafiltration buffer and the third diafiltration buffer contain the same excipients, and each excipient in the first diafiltration buffer is present in the third diafiltration buffer in an amount that is in the range of 90% to 110% of its amount in the third diafiltration buffer.
   E50. The method of any one of E46-E49, wherein the first diafiltration buffer and the third diafiltration buffer have substantially the same composition.
   E51. The method of any one of E46-E50, wherein the first diafiltration buffer, the second diafiltration buffer, and the third diafiltration buffer all have substantially the same composition.
   E52. A method for preparing a formulation comprising a recombinant protein, the method comprising diafiltering a first composition comprising a recombinant protein with a first diafiltration buffer and a second diafiltration buffer to obtain a second composition, wherein the first diafiltration buffer is a concentrate of the second diafiltration buffer.
   E53. The method of E52, wherein the first diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the second diafiltration buffer.
   E54. The method of E52 or E43, wherein the first diafiltration buffer is a 2× concentrate of the second diafiltration buffer.
   E55. The method of any one of E52-E54, wherein the diafiltering comprises diafiltering with the first diafiltration buffer for 0.5 to 3 diavolumes followed by diafiltering with the second diafiltration buffer for 6.5 to 9.5 diavolumes.
   E56. The method of any one of E52-E55, wherein the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is at most 10.
   E57. The method of any one of E52-E56, wherein the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is 10.
   E58. The method of any one of E52-E57, wherein the diafiltering comprises diafiltering with the first diafiltration buffer for 0.5 diavolumes followed by diafiltering with the second diafiltration buffer for 6.5 to 9.5 diavolumes.
   E59. The method of any one of E52-E57, wherein the diafiltering comprises diafiltering with the first diafiltration buffer for one diavolume followed by diafiltering with the second diafiltration buffer for 6 to 9 diavolumes.
   E60. The method of any one of E52-E57, wherein the diafiltering comprises diafiltering with the first diafiltration buffer for two diavolumes followed by diafiltering with the second diafiltration buffer for 5 to 8 diavolumes.
   E61. The method of any one of E52-E57, wherein the diafiltering comprises diafiltering with the first diafiltration buffer for three diavolumes followed by diafiltering with the second diafiltration buffer for 4 to 7 diavolumes.
   E62. The method of any one of E52-E61, wherein the recombinant protein is present in the first composition at a concentration that is at least 100% of a concentration of the recombinant protein in the second composition.
   E63. The method of any one of E52-E62, wherein the recombinant protein is present in the first composition at a concentration that is at least 120% of a concentration of the recombinant protein in the second composition.
   E64. The method of any one of E52-E63, wherein the recombinant protein is present in the first composition at a concentration that is at least 140% of a concentration of the recombinant protein in the second composition.
   E65. The method of any one of E52-E62, wherein the recombinant protein is present in the first composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition.
   E66. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with a second diafiltration buffer and a third diafiltration buffer to obtain a second composition, wherein the second diafiltration buffer is a concentrate of the third diafiltration buffer.
   E67. The method of E66, wherein the first diafiltration buffer and the third diafiltration buffer contain the same excipients, and each excipient in the first diafiltration buffer is present in the third diafiltration buffer in an amount that is in the range of 90% to 110% of its amount in the third diafiltration buffer.
   E68. The method of E66 or E67, wherein the first diafiltration buffer and the third diafiltration buffer have substantially the same composition.
   E69. The method of any one of E66-E68, wherein the second diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the third diafiltration buffer.
   E70. The method of any one of E66-E69, wherein the second diafiltration buffer is a 2× concentrate of the third diafiltration buffer.
   E71. The method of any one of any one of E66-E70, wherein the diafiltering comprises diafiltering with the second diafiltration buffer for 0.5 to 3 diavolumes followed by diafiltering with the third diafiltration buffer for 6.5 to 9.5 diavolumes.
   E72. The method of any one of E66-E71, wherein the diafiltering comprises diafiltering with the second diafiltration buffer for 0.5 to 3 diavolumes followed by diafiltering with the third diafiltration buffer for 6.5 to 9.5 diavolumes, wherein the total number of diavolumes used during the diafiltering is at most 10.
   E73. The method of any one of E66-E72, wherein the diafiltering comprises diafiltering with the second diafiltration buffer for 0.5 to 3 diavolumes followed by diafiltering with the third diafiltration buffer for 6.5 to 9.5 diavolumes, wherein the total number of diavolumes used during the diafiltering is 10.
   E74. The method of any one of E66-E73, wherein the diafiltering comprises diafiltering with the second diafiltration buffer for 0.5 diavolumes followed by diafiltering with the third diafiltration buffer for 6.5 to 9.5 diavolumes.
   E75. The method of any one of E66-E73, wherein the diafiltering comprises diafiltering with the second diafiltration buffer for one diavolume followed by diafiltering with the third diafiltration buffer for 6 to 9 diavolumes.
   E76. The method of any one of E66-E73, wherein the diafiltering comprises diafiltering with the second diafiltration buffer for two diavolumes followed by diafiltering with the third diafiltration buffer for 5 to 8 diavolumes.
   E77. The method of any one of E66-E73, wherein the diafiltering comprises diafiltering with the second diafiltration buffer for three diavolumes followed by diafiltering with the third diafiltration buffer for 4 to 7 diavolumes.
   E78. The method of any one of E66-E77, wherein the recombinant protein is present in the first concentrated composition at a concentration that is at least 100% of a concentration of the recombinant protein in the second composition.
   E79. The method of any one of E66-E78, wherein the recombinant protein is present in the first concentrated composition at a concentration that is at least 120% of a concentration of the recombinant protein in the second composition.
   E80. The method of any one of E66-E79, wherein the recombinant protein is present in the first concentrated composition at a concentration that is at least 140% of a concentration of the recombinant protein in the second composition.
   E81. The method of any one of E66-E78, wherein the recombinant protein is present in the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition.
   E82. A method for preparing a formulation comprising a recombinant protein, the method comprising:
concentrating a first composition comprising a recombinant protein by ultrafiltration to obtain a first concentrated composition; and
diafiltering the first concentrated composition to obtain a second composition, wherein the recombinant protein is present in the first concentrated composition at a concentration that is at least 120% of a concentration of the recombinant protein in the second composition.
   E83. A method for preparing a formulation comprising a recombinant protein, the method comprising:
concentrating a first composition comprising a recombinant protein by ultrafiltration to obtain a first concentrated composition; and
diafiltering the first concentrated composition with a first diafiltration buffer and a second diafiltration buffer to obtain a second composition, wherein:
the first diafiltration buffer is a concentrate of the second diafiltration buffer; and
the recombinant protein is present in the first concentrated composition at a concentration that is at least 120% of a concentration of the recombinant protein in the second composition.
   E84. The method of E83, wherein the first diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the second diafiltration buffer.
   E85. The method of E83 or E84, wherein the first diafiltration buffer is a 2× concentrate of the second diafiltration buffer.
   E86. The method of any one of E83-E85, wherein the diafiltering comprises diafiltering with the first diafiltration buffer for 0.5 to 3 diavolumes followed by diafiltering with the second diafiltration buffer for 6.5 to 9.5 diavolumes.
   E87. The method of any one of E83-E86, wherein the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is at most 10.
   E88. The method of any one of E83-E87, wherein the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is 10.
   E89. The method of any one of E83-E88, wherein the diafiltering comprises diafiltering with the first diafiltration buffer for 0.5 diavolumes followed by diafiltering with the second diafiltration buffer for 6.5 to 9.5 diavolumes.
   E90. The method of any one of E83-E88, wherein the diafiltering comprises diafiltering with the first diafiltration buffer for one diavolume followed by diafiltering with the second diafiltration buffer for 6 to 9 diavolumes.
   E91. The method of any one of E83-E88, wherein the diafiltering comprises diafiltering with the first diafiltration buffer for two diavolumes followed by diafiltering with the second diafiltration buffer for 5 to 8 diavolumes.
   E92. The method of any one of E83-E88, wherein the diafiltering comprises diafiltering with the first diafiltration buffer for three diavolumes followed by diafiltering with the second diafiltration buffer for 4 to 7 diavolumes.
   E93. The method of any one of E82-E92, wherein the first concentrated composition is diluted with a diafiltration buffer prior to the diafiltering.
   E94. The method of any one of E82-E93, wherein the first concentrated composition is diluted with a diafiltration buffer such that the concentration of the recombinant protein in the composition post-dilution is substantially the same as the concentration in the second composition.
   E95. The method of any one of E82-E94, wherein the recombinant protein is present in the first concentrated composition at a concentration that is at least 140% of a concentration of the recombinant protein in the second composition and the first concentrated composition is optionally diluted with a diafiltration buffer prior to the diafiltering, preferably wherein the first concentrated composition is diluted such that the concentration of the recombinant protein in the composition post-dilution is substantially the same as the concentration in the second composition.
   E96. The method of any one of E82-E93, wherein the recombinant protein is present in the first concentrated composition at a concentration that is in the range of 120% to 150% (e.g., 120% to 140%; 120%; 125%; 130%; 135%; 140%; 145%; 150%) of a concentration of the recombinant protein in the second composition and the first concentrated composition is optionally diluted with a diafiltration buffer prior to the diafiltering, preferably wherein the first concentrated composition is diluted such that the concentration of the recombinant protein in the composition post-dilution is substantially the same as the concentration in the second composition.
   E97. The method of any one of E1-E96, wherein the recombinant protein is an antibody.
   E98. The method of any one of E1-E96, wherein the recombinant protein is a human antibody.
   E99. The method of any one of E1-E96, wherein the recombinant protein is an IgG1, IgG2, or IgG4 antibody.
   E100. The method of any one of E1-E96, wherein the recombinant protein is a human IgG1, IgG2, or IgG4 antibody.
   E101. The method of any one of E1-E96, wherein the recombinant protein is an antibody fragment.
   E102. The method of any one of E1-E97, wherein the recombinant protein is a fusion protein.

### Ultrafiltration/Diafiltration Processes

Provided herein is a method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer to obtain a second composition,
wherein the first diafiltration buffer is prepared by diluting a diafiltration buffer concentrate.

In some embodiments, the diafiltering comprises diafiltering the first concentrated composition with 6 to 10 (such as, e.g., 7 to 10; 6 to 9; 6; 7; 8, 9; 10) diavolumes of the first diafiltration buffer. In some embodiments, the diafiltering comprises diafiltering the first concentrated composition with 7 to 10 diavolumes of the first diafiltration buffer. In some embodiments, the diafiltering comprises diafiltering the first concentrated composition with 8 to 10 diavolumes of the first diafiltration buffer.

In some embodiments, the diafiltering comprises diafiltering the first concentrated composition with 6 diavolumes of the first diafiltration buffer. In some embodiments, the diafiltering comprises diafiltering the first concentrated composition with 7 diavolumes of the first diafiltration buffer. In some embodiments, the diafiltering comprises diafiltering the first concentrated composition with 8 diavolumes of the first diafiltration buffer. In some embodiments, the diafiltering comprises diafiltering the first concentrated composition with 9 diavolumes of the first diafiltration buffer. In some embodiments, the diafiltering comprises diafiltering the first concentrated composition with 10 diavolumes of the first diafiltration buffer.

In some embodiments, the diluting is performed using an in-line dilution system. In some embodiments, the diluting is performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the diluting is continuously performed using an in-line dilution system. In some embodiments, the diluting is continuously performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the first diafiltration buffer is continuously prepared by diluting a diafiltration buffer concentrate at an intended dilution ratio of 1:1 with water (e.g., deionized water) using an in-line dilution system. In some embodiments, the first diafiltration buffer is continuously prepared by diluting a diafiltration buffer concentrate at an intended dilution ratio of 1:1 with water (e.g., deionized water) using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the recombinant protein is present in the first concentrated composition at a concentration that is at least 100% (e.g., at least 105%; at least 110%; at least 115%; at least 120%; at least 125%; at least 130%; at least 135%; at least 140%; 100%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, the recombinant protein is present in the first concentrated composition at a concentration that is at least 120% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, the recombinant protein is present in the first concentrated composition at a concentration that is at least 140% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, the recombinant protein is present in the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, no other step of the method uses a diafiltration buffer prepared by dilution from a diafiltration buffer concentrate.

In some embodiments, the method further comprises:
concentrating the second composition to obtain a second concentrated composition;
flushing the equilibrated UF/DF membrane filter with a second diafiltration buffer to obtain a recovery composition, wherein the second diafiltration buffer is not prepared by diluting a diafiltration buffer concentrate; and
combining the recovery composition and the second concentrated composition to obtain a third composition.

In some embodiments, concentrating the second composition comprises ultrafiltering using the equilibrated UF/DF membrane filter.

In some embodiments, the first diafiltration buffer and the second diafiltration buffer contain the same excipients, and each excipient in the first diafiltration buffer is present in the second diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the second diafiltration buffer.

In some embodiments, the first diafiltration buffer and the second diafiltration buffer have substantially the same composition.

In some embodiments, the second diafiltration buffer is batch prepared at its intended composition.

In other embodiments, the method further comprises:
flushing the equilibrated UF/DF membrane filter with a second diafiltration buffer to obtain a recovery composition, wherein the second diafiltration buffer is not prepared by diluting a diafiltration buffer concentrate; and
combining the recovery composition and the second composition to obtain a third composition.

In some embodiments, the first diafiltration buffer and the second diafiltration buffer contain the same excipients, and each excipient in the first diafiltration buffer is present in the second diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the second diafiltration buffer.

In some embodiments, the first diafiltration buffer and the second diafiltration buffer have substantially the same composition.

In some embodiments, the second diafiltration buffer is batch prepared at its intended composition.

In some embodiments, the recombinant protein is an antigen-binding protein. In some embodiments, the recombinant protein is an antibody or an antibody fragment. In some embodiments, the recombinant protein is an antibody. In some embodiments, the recombinant protein is an antibody fragment.

In some embodiments, the recombinant protein is an IgG1, IgG2, or IgG4 antibody. In some embodiments, the recombinant protein is an IgG1 antibody. In some embodiments, the recombinant protein is an IgG2 antibody. In some embodiments, the recombinant protein is an IgG4 antibody.

In some embodiments, the recombinant protein is a human antibody. In some embodiments, the recombinant protein is a human IgG1, IgG2, or IgG4 antibody. In some embodiments, the recombinant protein is a human IgG1 antibody. In some embodiments, the recombinant protein is a human IgG2 antibody. In some embodiments, the recombinant protein is a human IgG4 antibody.

In some embodiments, the recombinant protein is a fragment of an IgG1, IgG2, or IgG4 antibody. In some embodiments, the recombinant protein is a fragment of an IgG1 antibody. In some embodiments, the recombinant protein is a fragment of an IgG2 antibody. In some embodiments, the recombinant protein is a fragment of an IgG4 antibody.

In some embodiments, the recombinant protein is a fragment of a human antibody. In some embodiments, the recombinant protein is a fragment of a human IgG1, IgG2, or IgG4 antibody. In some embodiments, the recombinant protein is a fragment of a human IgG1 antibody. In some embodiments, the recombinant protein is a fragment of a human IgG2 antibody. In some embodiments, the recombinant protein is a fragment of a human IgG4 antibody.

In some embodiments, the recombinant protein is a fusion protein.

Also provided herein is a method for preparing a formulation comprising a recombinant protein, the method comprising diafiltering a first composition comprising a recombinant protein with a first diafiltration buffer and a second diafiltration buffer to obtain a second composition, wherein the first diafiltration buffer is prepared by diluting a diafiltration buffer concentrate, and the second diafiltration buffer is not prepared by diluting a diafiltration buffer concentrate. For example, in some embodiments, the second diafiltration buffer is batch prepared at its intended composition.

In some embodiments, the first diafiltration buffer and the second diafiltration buffer contain the same excipients, and each excipient in the first diafiltration buffer is present in the second diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the second diafiltration buffer.

In some embodiments, the first diafiltration buffer and the second diafiltration buffer have substantially the same composition.

In some embodiments, the diafiltering comprises diafiltering with the first diafiltration buffer for 6 to 6.5 diavolumes followed by diafiltering with the second diafiltration buffer for 0.5 to 1 diavolumes.

In some embodiments, the diafiltering comprises diafiltering with the first diafiltration buffer for 6 to 6.5 diavolumes followed by diafiltering with the second diafiltration buffer for 0.5 to 1 diavolumes, wherein the total number of diavolumes used during the diafiltering is 7. In some embodiments, the diafiltering comprises diafiltering with the first diafiltration buffer for 6 diavolumes followed by diafiltering with the second diafiltration buffer for 1 diavolume. In some embodiments, the diafiltering comprises diafiltering with the first diafiltration buffer for 6.5 diavolumes followed by diafiltering with the second diafiltration buffer for 0.5 diavolumes.

In some embodiments, the diluting is performed using an in-line dilution system. In some embodiments, the diluting is performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the diluting is continuously performed using an in-line dilution system. In some embodiments, the diluting is continuously performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the first diafiltration buffer is continuously prepared by diluting a diafiltration buffer concentrate at an intended dilution ratio of 1:1 with water (e.g., deionized water) using an in-line dilution system. In some embodiments, the first diafiltration buffer is continuously prepared by diluting a diafiltration buffer concentrate at an intended dilution ratio of 1:1 with water (e.g., deionized water) using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, prior to the diafiltering, the recombinant protein is present in the first concentrated composition at a concentration that is at least 100% (e.g., at least 105%; at least 110%; at least 115%; at least 120%; at least 125%; at least 130%; at least 135%; at least 140%; 100%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first composition at a concentration that is at least 120% of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, prior to the diafiltering, the recombinant protein is present in the first composition at a concentration that is at least 140% of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, prior to the diafiltering, the recombinant protein is present in the first composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, no other step of the method uses a diafiltration buffer prepared by dilution from a diafiltration buffer concentrate.

In some embodiments, the method further comprises:
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second composition to obtain a third composition.

In other embodiments, the method further comprises:
concentrating the second composition to obtain a second concentrated composition;
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second concentrated composition to obtain a third composition.

In some embodiments, concentrating the second composition comprises ultrafiltering using the equilibrated UF/DF membrane filter.

In some embodiments, the first diafiltration buffer and the third diafiltration buffer contain the same excipients, and each excipient in the first diafiltration buffer is present in the third diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the third diafiltration buffer.

In some embodiments, the first diafiltration buffer and the third diafiltration buffer have substantially the same composition. In some embodiments, the first diafiltration buffer, the second diafiltration buffer, and the third diafiltration buffer all have substantially the same composition.

In some embodiments, the recombinant protein is an antigen-binding protein. In some embodiments, the recombinant protein is an antibody or an antibody fragment. In some embodiments, the recombinant protein is an antibody. In some embodiments, the recombinant protein is an antibody fragment.

In some embodiments, the recombinant protein is an IgG1, IgG2, or IgG4 antibody. In some embodiments, the recombinant protein is an IgG1 antibody. In some embodiments, the recombinant protein is an IgG2 antibody. In some embodiments, the recombinant protein is an IgG4 antibody.

In some embodiments, the recombinant protein is a human antibody. In some embodiments, the recombinant protein is a human IgG1, IgG2, or IgG4 antibody. In some embodiments, the recombinant protein is a human IgG1 antibody. In some embodiments, the recombinant protein is a human IgG2 antibody. In some embodiments, the recombinant protein is a human IgG4 antibody.

In some embodiments, the recombinant protein is a fragment of an IgG1, IgG2, or IgG4 antibody. In some embodiments, the recombinant protein is a fragment of an IgG1 antibody. In some embodiments, the recombinant protein is a fragment of an IgG2 antibody. In some embodiments, the recombinant protein is a fragment of an IgG4 antibody.

In some embodiments, the recombinant protein is a fragment of a human antibody. In some embodiments, the recombinant protein is a fragment of a human IgG1, IgG2, or IgG4 antibody. In some embodiments, the recombinant protein is a fragment of a human IgG1 antibody. In some embodiments, the recombinant protein is a fragment of a human IgG2 antibody. In some embodiments, the recombinant protein is a fragment of a human IgG4 antibody.

In some embodiments, the recombinant protein is a fusion protein.

Also provided herein is a method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer and a second diafiltration buffer to obtain a second composition,
wherein:
   the first diafiltration buffer is prepared by diluting a diafiltration buffer concentrate; and
   the second diafiltration buffer is not prepared by diluting a diafiltration buffer concentrate.

In some embodiments, the second diafiltration buffer is batch prepared at its intended composition.

In some embodiments, the first diafiltration buffer and the second diafiltration buffer contain the same excipients, and each excipient in the first diafiltration buffer is present in the second diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the second diafiltration buffer.

In some embodiments, the first diafiltration buffer and the second diafiltration buffer have substantially the same composition.

In some embodiments, the diafiltering comprises diafiltering with the first diafiltration buffer for 6 to 6.5 diavolumes followed by diafiltering with the second diafiltration buffer for 0.5 to 1 diavolumes.

In some embodiments, the diafiltering comprises diafiltering with the first diafiltration buffer for 6 to 6.5 diavolumes followed by diafiltering with the second diafiltration buffer for 0.5 to 1 diavolumes, wherein the total number of diavolumes used during the diafiltering is 7. In some embodiments, the diafiltering comprises diafiltering with the first diafiltration buffer for 6 diavolumes followed by diafiltering with the second diafiltration buffer for 1 diavolume. In some embodiments, the diafiltering comprises diafiltering with the first diafiltration buffer for 6.5 diavolumes followed by diafiltering with the second diafiltration buffer for 0.5 diavolumes.

In some embodiments, the diluting is performed using an in-line dilution system. In some embodiments, the diluting is performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the diluting is continuously performed using an in-line dilution system. In some embodiments, the diluting is continuously performed using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, the first diafiltration buffer is prepared by diluting a diafiltration buffer concentrate with water (e.g., deionized water). In some embodiments, the first diafiltration buffer is prepared by diluting a diafiltration buffer concentrate at an intended dilution ratio of 1:1. In some embodiments, the first diafiltration buffer is prepared by diluting a diafiltration buffer concentrate at an intended dilution ratio of 1:1 with water (e.g., deionized water).

In some embodiments, the first diafiltration buffer is continuously prepared by diluting a diafiltration buffer concentrate at an intended dilution ratio of 1:1 with water (e.g., deionized water) using an in-line dilution system. In some embodiments, the first diafiltration buffer is continuously prepared by diluting a diafiltration buffer concentrate at an intended dilution ratio of 1:1 with water (e.g., deionized water) using an in-line dilution system, wherein a pump of the in-line dilution system has a relative dilution accuracy in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of an intended dilution ratio.

In some embodiments, prior to the diafiltering, the recombinant protein is present in the first concentrated composition at a concentration that is at least 100% (e.g., at least 105%; at least 110%; at least 115%; at least 120%; at least 125%; at least 130%; at least 135%; at least 140%; 100%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is at least 120% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is at least 140% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, no other step of the method uses a diafiltration buffer prepared by dilution from a diafiltration buffer concentrate.

In some embodiments, the method further comprises:
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second composition to obtain a third composition.

In other embodiments, the method further comprises:
concentrating the second composition to obtain a second concentrated composition;
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second concentrated composition to obtain a third composition.

In some embodiments, concentrating the second composition comprises ultrafiltering using the equilibrated UF/DF membrane filter.

In some embodiments, the first diafiltration buffer and the third diafiltration buffer contain the same excipients, and each excipient in the first diafiltration buffer is present in the third diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the third diafiltration buffer.

In some embodiments, the first diafiltration buffer and the third diafiltration buffer have substantially the same composition. In some embodiments, the first diafiltration buffer, the second diafiltration buffer, and the third diafiltration buffer all have substantially the same composition.

In some embodiments, the recombinant protein is an antigen-binding protein. In some embodiments, the recombinant protein is an antibody or an antibody fragment. In some embodiments, the recombinant protein is an antibody. In some embodiments, the recombinant protein is an antibody fragment.

In some embodiments, the recombinant protein is an IgG1, IgG2, or IgG4 antibody. In some embodiments, the recombinant protein is an IgG1 antibody. In some embodiments, the recombinant protein is an IgG2 antibody. In some embodiments, the recombinant protein is an IgG4 antibody.

In some embodiments, the recombinant protein is a human antibody. In some embodiments, the recombinant protein is a human IgG1, IgG2, or IgG4 antibody. In some embodiments, the recombinant protein is a human IgG1 antibody. In some embodiments, the recombinant protein is a human IgG2 antibody. In some embodiments, the recombinant protein is a human IgG4 antibody.

In some embodiments, the recombinant protein is a fragment of an IgG1, IgG2, or IgG4 antibody. In some embodiments, the recombinant protein is a fragment of an IgG1 antibody. In some embodiments, the recombinant protein is a fragment of an IgG2 antibody. In some embodiments, the recombinant protein is a fragment of an IgG4 antibody.

In some embodiments, the recombinant protein is a fragment of a human antibody. In some embodiments, the recombinant protein is a fragment of a human IgG1, IgG2, or IgG4 antibody. In some embodiments, the recombinant protein is a fragment of a human IgG1 antibody. In some embodiments, the recombinant protein is a fragment of a human IgG2 antibody. In some embodiments, the recombinant protein is a fragment of a human IgG4 antibody.

In some embodiments, the recombinant protein is a fusion protein.

Also provided herein is a method for preparing a formulation comprising a recombinant protein, the method comprising diafiltering a first composition comprising a recombinant protein with a first diafiltration buffer and a second diafiltration buffer to obtain a second composition, wherein the first diafiltration buffer is a concentrate of the second diafiltration buffer.

In some embodiments, the first diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the second diafiltration buffer. In some embodiments, the first diafiltration buffer is a 2× concentrate of the second diafiltration buffer. In some embodiments, the first diafiltration buffer is a 3× concentrate of the second diafiltration buffer. In some embodiments, the first diafiltration buffer is a 4× concentrate of the second diafiltration buffer. In some embodiments, the first diafiltration buffer is a 5× concentrate of the second diafiltration buffer.

In some embodiments, the diafiltering comprises diafiltering with the first diafiltration buffer for 0.5 to 3 diavolumes followed by diafiltering with the second diafiltration buffer for 6.5 to 9.5 diavolumes. In some embodiments, the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is at most 10. In some embodiments, the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is 10.

In some embodiments, the diafiltering comprises diafiltering with the first diafiltration buffer for 0.5 diavolumes followed by diafiltering with the second diafiltration buffer for 6.5 to 9.5 diavolumes. In some embodiments, the diafiltering comprises diafiltering with the first diafiltration buffer for 0.5 diavolumes followed by diafiltering with the second diafiltration buffer for 6.5 diavolumes. In some embodiments, the diafiltering comprises diafiltering with the first diafiltration buffer for 0.5 diavolumes followed by diafiltering with the second diafiltration buffer for 7.5 diavolumes. In some embodiments, the diafiltering comprises diafiltering with the first diafiltration buffer for 0.5 diavolumes followed by diafiltering with the second diafiltration buffer for 8.5 diavolumes. In some embodiments, the diafiltering comprises diafiltering with the first diafiltration buffer for 0.5 diavolumes followed by diafiltering with the second diafiltration buffer for 9.5 diavolumes.

In some embodiments, the diafiltering comprises diafiltering with the first diafiltration buffer for one diavolume followed by diafiltering with the second diafiltration buffer for 6 to 9 diavolumes. In some embodiments, the diafiltering comprises diafiltering with the first diafiltration buffer for one diavolume followed by diafiltering with the second diafiltration buffer for 6 diavolumes. In some embodiments, the diafiltering comprises diafiltering with the first diafiltration buffer for one diavolume followed by diafiltering with the second diafiltration buffer for 7 diavolumes. In some embodiments, the diafiltering comprises diafiltering with the first diafiltration buffer for one diavolume followed by diafiltering with the second diafiltration buffer for 8 diavolumes. In some embodiments, the diafiltering comprises diafiltering with the first diafiltration buffer for one diavolume followed by diafiltering with the second diafiltration buffer for 9 diavolumes.

In some embodiments, the diafiltering comprises diafiltering with the first diafiltration buffer for two diavolumes followed by diafiltering with the second diafiltration buffer for 5 to 8 diavolumes. In some embodiments, the diafiltering comprises diafiltering with the first diafiltration buffer for two diavolumes followed by diafiltering with the second diafiltration buffer for 5 diavolumes. In some embodiments, the diafiltering comprises diafiltering with the first diafiltration buffer for two diavolumes followed by diafiltering with the second diafiltration buffer for 6 diavolumes. In some embodiments, the diafiltering comprises diafiltering with the first diafiltration buffer for two diavolumes followed by diafiltering with the second diafiltration buffer for 7 diavolumes. In some embodiments, the diafiltering comprises diafiltering with the first diafiltration buffer for two diavolumes followed by diafiltering with the second diafiltration buffer for 8 diavolumes.

In some embodiments, the diafiltering comprises diafiltering with the first diafiltration buffer for three diavolumes followed by diafiltering with the second diafiltration buffer for 4 to 7 diavolumes. In some embodiments, the diafiltering comprises diafiltering with the first diafiltration buffer for three diavolumes followed by diafiltering with the second diafiltration buffer for 4 diavolumes. In some embodiments, the diafiltering comprises diafiltering with the first diafiltration buffer for three diavolumes followed by diafiltering with the second diafiltration buffer for 5 diavolumes. In some embodiments, the diafiltering comprises diafiltering with the first diafiltration buffer for three diavolumes followed by diafiltering with the second diafiltration buffer for 6 diavolumes. In some embodiments, the diafiltering comprises diafiltering with the first diafiltration buffer for three diavolumes followed by diafiltering with the second diafiltration buffer for 7 diavolumes.

In some embodiments, prior to the diafiltering, the recombinant protein is present in the first composition at a concentration that is at least 100% (e.g., at least 105%; at least 110%; at least 115%; at least 120%; at least 125%; at least 130%; at least 135%; at least 140%; 100%; 105%; 110%; 115%; 120%; 125%; 130%; 135%; 140%; 145%; 150%) of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, prior to the diafiltering, the recombinant protein is present in the first composition at a concentration that is at least 120% of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, prior to the diafiltering, the recombinant protein is present in the first composition at a concentration that is at least 140% of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, prior to the diafiltering, the recombinant protein is present in the first composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first composition is optionally diluted with a diafiltration buffer prior to the diafiltering. In some embodiments, the first composition is diluted such that the concentration of the recombinant protein in the composition post-dilution is substantially the same as the concentration in the second composition.

In some embodiments, the recombinant protein is an antigen-binding protein. In some embodiments, the recombinant protein is an antibody or an antibody fragment. In some embodiments, the recombinant protein is an antibody. In some embodiments, the recombinant protein is an antibody fragment.

In some embodiments, the recombinant protein is an IgG1, IgG2, or IgG4 antibody. In some embodiments, the recombinant protein is an IgG1 antibody. In some embodiments, the recombinant protein is an IgG2 antibody. In some embodiments, the recombinant protein is an IgG4 antibody.

In some embodiments, the recombinant protein is a human antibody. In some embodiments, the recombinant protein is a human IgG1, IgG2, or IgG4 antibody. In some embodiments, the recombinant protein is a human IgG1 antibody. In some embodiments, the recombinant protein is a human IgG2 antibody. In some embodiments, the recombinant protein is a human IgG4 antibody.

In some embodiments, the recombinant protein is a fragment of an IgG1, IgG2, or IgG4 antibody. In some embodiments, the recombinant protein is a fragment of an IgG1 antibody. In some embodiments, the recombinant protein is a fragment of an IgG2 antibody. In some embodiments, the recombinant protein is a fragment of an IgG4 antibody.

In some embodiments, the recombinant protein is a fragment of a human antibody. In some embodiments, the recombinant protein is a fragment of a human IgG1, IgG2, or IgG4 antibody. In some embodiments, the recombinant protein is a fragment of a human IgG1 antibody. In some embodiments, the recombinant protein is a fragment of a human IgG2 antibody. In some embodiments, the recombinant protein is a fragment of a human IgG4 antibody.

In some embodiments, the recombinant protein is a fusion protein.

Also provided herein is a method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with a second diafiltration buffer and a third diafiltration buffer to obtain a second composition, wherein the second diafiltration buffer is a concentrate of the third diafiltration buffer.

In some embodiments, the first diafiltration buffer and the third diafiltration buffer contain the same excipients, and each excipient in the first diafiltration buffer is present in the third diafiltration buffer in an amount that is in the range of 90% to 110% (e.g., 95% to 105%; 90%; 91%; 92%; 93%; 94%; 95%; 96%; 97%; 98%; 99%; 100%; 101%; 102%; 103%; 104%; 105%; 106%; 107%; 108%; 109%; 110%) of its amount in the third diafiltration buffer.

In some embodiments, the first diafiltration buffer and the third diafiltration buffer have substantially the same composition.

In some embodiments, the second diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the third diafiltration buffer. In some embodiments, the second diafiltration buffer is a 2× concentrate of the third diafiltration buffer. In some embodiments, the second diafiltration buffer is a 3× concentrate of the third diafiltration buffer. In some embodiments, the second diafiltration buffer is a 4× concentrate of the third diafiltration buffer. In some embodiments, the second diafiltration buffer is a 5× concentrate of the third diafiltration buffer.

In some embodiments, the diafiltering comprises diafiltering with the second diafiltration buffer for 0.5 to 3 diavolumes followed by diafiltering with the third diafiltration buffer for 6.5 to 9.5 diavolumes. In some embodiments, the diafiltering comprises diafiltering with the second diafiltration buffer for 0.5 to 3 diavolumes followed by diafiltering with the third diafiltration buffer for 6.5 to 9.5 diavolumes, wherein the total number of diavolumes used during the diafiltering is at most 10. In some embodiments, the diafiltering comprises diafiltering with the second diafiltration buffer for 0.5 to 3 diavolumes followed by diafiltering with the third diafiltration buffer for 6.5 to 9.5 diavolumes, wherein the total number of diavolumes used during the diafiltering is 10.

In some embodiments, the diafiltering comprises diafiltering with the second diafiltration buffer for 0.5 diavolumes followed by diafiltering with the third diafiltration buffer for 6.5 to 9.5 diavolumes. In some embodiments, the diafiltering comprises diafiltering with the second diafiltration buffer for 0.5 diavolumes followed by diafiltering with the third diafiltration buffer for 6.5 diavolumes. In some embodiments, the diafiltering comprises diafiltering with the second diafiltration buffer for 0.5 diavolumes followed by diafiltering with the third diafiltration buffer for 7.5 diavolumes. In some embodiments, the diafiltering comprises diafiltering with the second diafiltration buffer for 0.5 diavolumes followed by diafiltering with the third diafiltration buffer for 8.5 diavolumes. In some embodiments, the diafiltering comprises diafiltering with the second diafiltration buffer for 0.5 diavolumes followed by diafiltering with the third diafiltration buffer for 9.5 diavolumes.

In some embodiments, the diafiltering comprises diafiltering with the second diafiltration buffer for one diavolume followed by diafiltering with the third diafiltration buffer for 6 to 9 diavolumes. In some embodiments, the diafiltering comprises diafiltering with the second diafiltration buffer for one diavolume followed by diafiltering with the third diafiltration buffer for 6 diavolumes. In some embodiments, the diafiltering comprises diafiltering with the second diafiltration buffer for one diavolume followed by diafiltering with the third diafiltration buffer for 7 diavolumes. In some embodiments, the diafiltering comprises diafiltering with the second diafiltration buffer for one diavolume followed by diafiltering with the third diafiltration buffer for 8 diavolumes. In some embodiments, the diafiltering comprises diafiltering with the second diafiltration buffer for one diavolume followed by diafiltering with the third diafiltration buffer for 9 diavolumes.

In some embodiments, the diafiltering comprises diafiltering with the second diafiltration buffer for two diavolumes followed by diafiltering with the third diafiltration buffer for 5 to 8 diavolumes. In some embodiments, the diafiltering comprises diafiltering with the second diafiltration buffer for two diavolumes followed by diafiltering with the third diafiltration buffer for 5 diavolumes. In some embodiments, the diafiltering comprises diafiltering with the second diafiltration buffer for two diavolumes followed by diafiltering with the third diafiltration buffer for 6 diavolumes. In some embodiments, the diafiltering comprises diafiltering with the second diafiltration buffer for two diavolumes followed by diafiltering with the third diafiltration buffer for 7 diavolumes. In some embodiments, the diafiltering comprises diafiltering with the second diafiltration buffer for two diavolumes followed by diafiltering with the third diafiltration buffer for 8 diavolumes.

In some embodiments, the diafiltering comprises diafiltering with the second diafiltration buffer for three diavolumes followed by diafiltering with the third diafiltration buffer for 4 to 7 diavolumes. In some embodiments, the diafiltering comprises diafiltering with the second diafiltration buffer for three diavolumes followed by diafiltering with the third diafiltration buffer for 4 diavolumes. In some embodiments, the diafiltering comprises diafiltering with the second diafiltration buffer for three diavolumes followed by diafiltering with the third diafiltration buffer for 5 diavolumes. In some embodiments, the diafiltering comprises diafiltering with the second diafiltration buffer for three diavolumes followed by diafiltering with the third diafiltration buffer for 6 diavolumes. In some embodiments, the diafiltering comprises diafiltering with the second diafiltration buffer for three diavolumes followed by diafiltering with the third diafiltration buffer for 7 diavolumes.

In some embodiments, prior to the diafiltering, the recombinant protein is present in the first concentrated composition at a concentration that is at least 100% (e.g., at least 105%; at least 110%; at least 115%; at least 120%; at least 125%; at least 130%; at least 135%; at least 140%; 100%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is at least 120% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is at least 140% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, prior to the diafiltering, the recombinant protein is present in the first concentrated composition at a concentration that is in the range of 100% to 150% (e.g., 110% to 140%; 120% to 140%; 105%; 110%; 115%; 120%; 125%; 130%; 135%; 140%; 145%; 150%) of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

Provided herein is a method for preparing a formulation comprising a recombinant protein, the method comprising:
concentrating a first composition comprising a recombinant protein by ultrafiltration to obtain a first concentrated composition; and
diafiltering the first concentrated composition to obtain a second composition, wherein the recombinant protein is present in the first concentrated composition at a concentration that is at least 120% of a concentration of the recombinant protein in the second composition.

Also provided herein is a method for preparing a formulation comprising a recombinant protein, the method comprising:
concentrating a first composition comprising a recombinant protein by ultrafiltration to obtain a first concentrated composition; and
diafiltering the first concentrated composition with a first diafiltration buffer and a second diafiltration buffer to obtain a second composition, wherein:
the first diafiltration buffer is a concentrate of the second diafiltration buffer; and
the recombinant protein is present in the first concentrated composition at a concentration that is at least 120% of a concentration of the recombinant protein in the second composition.

In some embodiments, the first diafiltration buffer is at least a 2× (e.g., at least a 2.5×, at least a 3×; 2×; 2.25×; 2.5×; 3×; 3.25×; 3.5×; 3.75×; 4×; 4.25×; 4.5×; 4.75×; 5×; 2× to 3×; 2× to 4×; 2× to 5×) concentrate of the second diafiltration buffer. In some embodiments, the first diafiltration buffer is a 2× concentrate of the second diafiltration buffer. In some embodiments, the first diafiltration buffer is a 3× concentrate of the second diafiltration buffer. In some embodiments, the first diafiltration buffer is a 4× concentrate of the second diafiltration buffer. In some embodiments, the first diafiltration buffer is a 5× concentrate of the second diafiltration buffer.

In some embodiments, the diafiltering comprises diafiltering with the first diafiltration buffer for 0.5 to 3 diavolumes followed by diafiltering with the second diafiltration buffer for 6.5 to 9.5 diavolumes. In some embodiments, the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is at most 10. In some embodiments, the diafiltering comprises diafiltering with the first diafiltration buffer for 0.5 to 3 diavolumes followed by diafiltering with the second diafiltration buffer for 6.5 to 9.5 diavolumes, wherein the total number of diavolumes used during the diafiltering is 10.

In some embodiments, the diafiltering comprises diafiltering with the first diafiltration buffer for 0.5 diavolumes followed by diafiltering with the second diafiltration buffer for 6.5 to 9.5 diavolumes. In some embodiments, the diafiltering comprises diafiltering with the first diafiltration buffer for 0.5 diavolumes followed by diafiltering with the second diafiltration buffer for 6.5 diavolumes. In some embodiments, the diafiltering comprises diafiltering with the first diafiltration buffer for 0.5 diavolumes followed by diafiltering with the second diafiltration buffer for 7.5 diavolumes. In some embodiments, the diafiltering comprises diafiltering with the first diafiltration buffer for 0.5 diavolumes followed by diafiltering with the second diafiltration buffer for 8.5 diavolumes. In some embodiments, the diafiltering comprises diafiltering with the first diafiltration buffer for 0.5 diavolumes followed by diafiltering with the second diafiltration buffer for 9.5 diavolumes.

In some embodiments, the diafiltering comprises diafiltering with the first diafiltration buffer for one diavolume followed by diafiltering with the second diafiltration buffer for 6 to 9 diavolumes. In some embodiments, the diafiltering comprises diafiltering with the first diafiltration buffer for one diavolume followed by diafiltering with the second diafiltration buffer for 6 diavolumes. In some embodiments, the diafiltering comprises diafiltering with the first diafiltration buffer for one diavolume followed by diafiltering with the second diafiltration buffer for 7 diavolumes. In some embodiments, the diafiltering comprises diafiltering with the first diafiltration buffer for one diavolume followed by diafiltering with the second diafiltration buffer for 8 diavolumes. In some embodiments, the diafiltering comprises diafiltering with the first diafiltration buffer for one diavolume followed by diafiltering with the second diafiltration buffer for 9 diavolumes.

In some embodiments, the diafiltering comprises diafiltering with the first diafiltration buffer for two diavolumes followed by diafiltering with the second diafiltration buffer for 5 to 8 diavolumes. In some embodiments, the diafiltering comprises diafiltering with the first diafiltration buffer for two diavolumes followed by diafiltering with the second diafiltration buffer for 5 diavolumes. In some embodiments, the diafiltering comprises diafiltering with the first diafiltration buffer for two diavolumes followed by diafiltering with the second diafiltration buffer for 6 diavolumes. In some embodiments, the diafiltering comprises diafiltering with the first diafiltration buffer for two diavolumes followed by diafiltering with the second diafiltration buffer for 7 diavolumes. In some embodiments, the diafiltering comprises diafiltering with the first diafiltration buffer for two diavolumes followed by diafiltering with the second diafiltration buffer for 8 diavolumes.

In some embodiments, the diafiltering comprises diafiltering with the first diafiltration buffer for three diavolumes followed by diafiltering with the second diafiltration buffer for 4 to 7 diavolumes. In some embodiments, the diafiltering comprises diafiltering with the first diafiltration buffer for three diavolumes followed by diafiltering with the second diafiltration buffer for 4 diavolumes. In some embodiments, the diafiltering comprises diafiltering with the first diafiltration buffer for three diavolumes followed by diafiltering with the second diafiltration buffer for 5 diavolumes. In some embodiments, the diafiltering comprises diafiltering with the first diafiltration buffer for three diavolumes followed by diafiltering with the second diafiltration buffer for 6 diavolumes. In some embodiments, the diafiltering comprises diafiltering with the first diafiltration buffer for three diavolumes followed by diafiltering with the second diafiltration buffer for 7 diavolumes.

In some embodiments, the first concentrated composition is diluted with a diafiltration buffer prior to the diafiltering. In some embodiments, the first concentrated composition is diluted with a diafiltration buffer such that the concentration of the recombinant protein in the composition post-dilution is substantially the same as the concentration in the second composition.

In some embodiments, the recombinant protein is present in the first concentrated composition at a concentration that is at least 140% of a concentration of the recombinant protein in the second composition and the first concentrated composition is optionally diluted with a diafiltration buffer prior to the diafiltering. In some embodiments, the first concentrated composition is diluted such that the concentration of the recombinant protein in the composition post-dilution is substantially the same as the concentration in the second composition.

In some embodiments, the recombinant protein is present in the first concentrated composition at a concentration that is in the range of 120% to 150% (e.g., 120% to 140%; 120%; 125%; 130%; 135%; 140%; 145%; 150%) of a concentration of the recombinant protein in the second composition and the first concentrated composition is diluted with a diafiltration buffer prior to the diafiltering. In some embodiments, the first concentrated composition is optionally diluted such that the concentration of the recombinant protein in the composition post-dilution is substantially the same as the concentration in the second composition.

In some embodiments, the recombinant protein is an antigen-binding protein. In some embodiments, the recombinant protein is an antibody or an antibody fragment. In some embodiments, the recombinant protein is an antibody. In some embodiments, the recombinant protein is an antibody fragment.

In some embodiments, the recombinant protein is an IgG1, IgG2, or IgG4 antibody. In some embodiments, the recombinant protein is an IgG1 antibody. In some embodiments, the recombinant protein is an IgG2 antibody. In some embodiments, the recombinant protein is an IgG4 antibody.

In some embodiments, the recombinant protein is a human antibody. In some embodiments, the recombinant protein is a human IgG1, IgG2, or IgG4 antibody. In some embodiments, the recombinant protein is a human IgG1 antibody. In some embodiments, the recombinant protein is a human IgG2 antibody. In some embodiments, the recombinant protein is a human IgG4 antibody.

In some embodiments, the recombinant protein is a fragment of an IgG1, IgG2, or IgG4 antibody. In some embodiments, the recombinant protein is a fragment of an IgG1 antibody. In some embodiments, the recombinant protein is a fragment of an IgG2 antibody. In some embodiments, the recombinant protein is a fragment of an IgG4 antibody.

In some embodiments, the recombinant protein is a fragment of a human antibody. In some embodiments, the recombinant protein is a fragment of a human IgG1, IgG2, or IgG4 antibody. In some embodiments, the recombinant protein is a fragment of a human IgG1 antibody. In some embodiments, the recombinant protein is a fragment of a human IgG2 antibody. In some embodiments, the recombinant protein is a fragment of a human IgG4 antibody.

In some embodiments, the recombinant protein is a fusion protein.

### Purification Processes Preceding UF/DF

Compositions subjected to the intensified UF/DF methods disclosed herein may derive from a cell culture process and may be subjected to one or more purification processes prior to UF/DF. In some embodiments, one or more purification processes (e.g., one or more unit operations described herein) may be continuous with an intensified UF/DF method described herein. In some embodiments, one or more purification processes (e.g., one or more unit operations described herein) may be connected with an intensified UF/DF method described herein. For example, a viral filtration unit operation may be continuous or connected with an intensified UF/DF method described herein.

Illustratively, in an example upstream process preceding an intensified UF/DF method described herein, expressed recombinant proteins may be secreted into the culture medium from which they can be recovered and/or collected. Harvest operations comprising an acid precipitation may be combined with additional harvest strategies, including centrifugation, such as, e.g., disk-stack centrifugation, intermittent discharge centrifugation, or continuous solid discharge centrifugation; filtration, including, e.g., tangential flow filtration, microfiltration, ultrafiltration, and depth filtration; precipitation/sedimentation methods, such as, e.g., flocculation; and chromatography media-based separations.

Additionally, post-harvest recovery technologies, such as, e.g., protein A purification of immunoglobulin and immunoglobulin-like biologics, as well as chromatography-based separations and polishing steps that include column and alternative modes of chromatographic separations by ion exchange chromatography (IEX), including anion exchange chromatography (AEX) and/or cation exchange chromatography (CEX), hydrophobic interaction chromatography (HIC), mixed modal or multimodal chromatography (MM), hydroxyapatite chromatography (HA), reverse-phase chromatography, size exclusion chromatography (SEC), gel filtration, or any other known form of chromatographic separation of biological and/or biochemical substances, may be employed prior to performing a UF/DF method described herein.

In some embodiments, recombinant protein recovered from the host cells or cell culture medium may be further purified or partially purified to remove cell culture media components, host cell proteins, or nucleic acids, or other process or product-related impurities by one or more unit operations, before or after virus filtration. One of ordinary skill in the art can select the appropriate unit operation(s) for further purification of a recombinant protein based on the characteristics of the recombinant protein to be purified, the characteristics of host cell from which the recombinant protein is expressed, and the composition of the culture medium in which the host cells were grown. Illustratively, in some embodiments, the recombinant protein is purified from the harvest permeate by one or more of flocculation, precipitation, centrifugation, depth filtration, affinity chromatography, size exclusion chromatography, ion exchange chromatography, mixed mode anion exchange chromatography, hydrophobic interaction chromatography, or hydroxyapatite chromatography.

A capture unit operation may include capture chromatography that makes use of resins and/or membranes containing agents that will bind to the recombinant protein of interest, for example, affinity chromatography, size exclusion chromatography, ion exchange chromatography, hydrophobic interaction chromatography (HIC), immobilized metal affinity chromatography (IMAC), and the like. Such chromatographic materials are known in the art and are commercially available. For instance, if the recombinant protein is an antibody or contains components derived from an antibody (e.g., a Fc domain), affinity chromatography using ligands such as Protein A, Protein G, Protein A/G, or Protein L may be employed as a capture chromatography unit operation to further purify the recombinant protein. In other embodiments, the recombinant protein of interest may comprise a polyhistidine tag at its amino or carboxyl terminus and subsequently purified using IMAC. Recombinant proteins can be engineered to include other purification tags, such as, e.g., a FLAG® tag or c-myc epitope and subsequently purified by affinity chromatography using a specific antibody directed to such tag or epitope.

Additional unit operations to inactivate, reduce, and/or eliminate viral contaminants may include filtration processes and/or adjusting solution conditions. One method for achieving viral inactivation is incubation at low pH (e.g., pH<4). A low pH viral inactivation operation can be followed with a neutralization unit operation that readjusts the virus inactivated solution to a pH more compatible with the requirements of the subsequent unit operations. A low pH viral inactivation operation may also be followed by filtration, such as depth filtration with an optional virus pre-filter, to remove any resulting turbidity or precipitation. Adjusting the temperature or chemical composition (e.g., use of detergents) can also be used to achieve viral inactivation.

Virus filters are polymeric membranes with various membrane chemistries and complex pore structures designed to retain viral particles. Virus filters are commercially available from a variety of manufacturers (e.g., MilliporeSigma, Sartorius, Pall, and Asahi Kasei) and include large virus filters and small virus filters. Large virus filters are designed to retain viruses larger than 60 nm, while small virus filters are designed to retain viruses larger than 20 nm. Virus filters can be used in normal flow filter (NFF) or a tangential flow filtration (TFF) mode. In either TFF mode or NFF mode, filtration is conducted under conditions to retain viral contaminants (e.g., a virus having a 20 to 100 nm diameter) on the membrane surface while permitting passage of a recombinant protein through the filter membrane.

Non-limiting examples of virus filters include those formed from regenerated cellulose (e.g., cuprammonium-regenerated cellulose), polyethersulfone, polyarylsulphones, polysulfone, polyimide, polyamide, polyvinylidenedifluoride (PVDF), and the like. For example, non-limiting examples of virus filters include VIRESOLVE® membranes and RETROPORE™ membranes available from EMD Millipore, Billerica, Mass. These can be supplied in either a cartridge (NFF) form, such as VIRESOLVE® NFP viral filters, or as cassettes (for TFF), such as PELLICON® cassettes, available from EMD Millipore, Billerica, Mass.

A further non-limiting example is the Sartorius Virosart® CPV small virus filter, which comprises a polyethersulfone membrane. Other PES membrane filters include, but are not limited to, Viresolve® NFR, Viresolve® Pro, Virosart® CPV, Virosart® HF, Virosart® HC, and Pegasus™ grade Prime. Additional non-limiting example filters include the Planova™ BioEX filter and the Viresolve® NFP filter, both of which comprise a polyvinylidene fluoride (PVDF) membrane, as well as Asahi Kasei's 15N, 20N, and 35N filters, which comprise a cuprammonium-regenerated cellulose membrane (e.g., a hollow fiber cuprammonium-regenerated cellulose membrane). Alternative examples of virus filters are known in the art and include hydrophilic acrylate-modified PVDF membranes from Pall, such as, e.g., Ultipor® VF grade DV20, Ultipor® VF grade DV50, and Pegasus™ grade PV4.

Virus prefilters, which are commercially available from several sources (e.g., MilliporeSigma, Sartorius, Pall, Asahi Kasei), remove protein aggregates and other process impurities that may otherwise foul virus filters. Common virus prefilters use a variety of membrane chemistries, such as a PES membrane surface modified via cross-linked polymeric sulfonic acid cation exchange chemistry (Viresolve® Shield, MilliporeSigma) and a PES membrane surface modified via cross-linked mixed-mode chemistry (Viresolve® Shield-H, MilliporeSigma). Additionally, depth filters composed of diatomaceous earth, cellulose fibers, and a binder comprising cationic imine groups (Viresolve® Prefilter, MilliporeSigma), pleated sheet membranes (Pegasus™ Protect, Pall; Pegasus™ grade UL6, Pall), and triple layer, flat sheet membranes (Virosart® Max, Sartorius) can be used as prefilters.

Virus prefilters commonly act at least in part through size exclusion, for example, with size exclusion cutoffs between about 75 nm and about 0.2 μm (e.g., 75 nm (Planova 75N, Asahi Kasei); 0.1 μm (Virosart® Max, Sartorius; Viresolve® Prefilter, MilliporeSigma); 0.2 μm (Viresolve® Shield, MilliporeSigma; Viresolve® Shield-H, MilliporeSigma; Pegasus™ Protect, Pall)).

A polishing unit operation may make use of various chromatographic methods for the purification of the protein of interest and clearance of contaminants and impurities. The polishing chromatography unit operation may make use of resins and/or membranes containing agents that can be used in either a “flow-through mode,” in which the protein of interest is contained in the eluent and the contaminants and impurities are bound to the chromatographic medium, or “bind and elute mode,” in which the protein of interest is bound to the chromatographic medium and eluted after the contaminants and impurities have flowed through or been washed off the chromatographic medium. Examples of such polish chromatography methods include, but are not limited to, ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography, and size-exclusion chromatography (e.g., gel filtration).

### Recombinant Proteins

Compositions comprising any type of recombinant protein, including proteins containing single polypeptide chains or multiple polypeptide chains, can be formulated according to the methods of the present disclosure. Such recombinant proteins include, but are not limited to, secreted proteins, non-secreted proteins, intracellular proteins, or membrane-bound proteins. Illustratively, recombinant proteins can include, but are not limited to, cytokines, growth factors, hormones, muteins, fusion proteins, antibodies, antibody fragments, peptibodies, T-cell engaging molecules, and multi-specific antigen binding proteins. In some embodiments, the recombinant protein is a fusion protein.

In other embodiments, the recombinant protein is an antigen-binding protein. Antigen-binding proteins include, but are not limited to, antibodies, peptibodies, antibody derivatives, antibody analogs, fusion proteins (including, e.g., single chain variable fragments (scFvs), double-chain (divalent) scFvs, and IgGscFv (see, e.g., Orcutt et al., 2010, Protein Eng Des Sel 23:221-228)), hetero-IgG molecules (see, e.g., Liu et al., 2015, J Biol Chem 290:7535-7562), muteins, and XmAb® (Xencor, Inc., Monrovia, CA). Additional antigen-binding proteins include, but are not limited to, bispecific T cell engagers (BiTE®), bispecific T cell engagers having extensions, such as, e.g., half-life extensions, such as, e.g., HLE BiTE molecules, Heterolg BITE molecules, and others, chimeric antigen receptors (CARs, CAR Ts), and T cell receptors (TCRs).

In some embodiments, the antigen-binding protein binds to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β01, TGF-β02, TGF-β03, TGF-β04, or TGF-β05, insulin-like growth factors-I and -II (IGF-I and IGF-II), des(1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDLI, mannose receptor/hCGβ, hepatitis-C virus, mesothelin dsFv[PE38] conjugate, Legionella pneumophila (11y), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In other embodiments, the recombinant protein is an antibody. In some embodiments, the antibody is a human antibody.

In some embodiments, the antibody is selected from abrilumab, brazikumab, brodalumab, crizanlizumab, denosumab, eculizumab, erenumab, evolocumab, fremanezumab, meplazumab, nemolizumab, ontamalimab, panitumumab, prezalumab, ravulizumab, rilotumumab, romosozumab, satralizumab, tafolecimab, tanezumab, tezepelumab, tremelimumab, utomilumab, and volagidemab. In some embodiments, the antibody is selected from denosumab, erenumab, evolocumab, panitumumab, romosozumab, and tezepelumab. In some embodiments, the antibody is denosumab. In some embodiments, the antibody is erenumab. In some embodiments, the antibody is evolocumab. In some embodiments, the antibody is panitumumab. In some embodiments, the antibody is romosozumab. In some embodiments, the antibody is tezepelumab.

In some embodiments, the antibody is an IgG1, IgG2, or IgG4 antibody. In some embodiments, the antibody is a human IgG1, IgG2, or IgG4 antibody.

In some embodiments, the antibody is an IgG1 antibody. In some embodiments, the antibody is a human IgG1 antibody.

In some embodiments, the antibody is an IgG2 antibody. In some embodiments, the antibody is a human IgG2 antibody.

In some embodiments, the antibody is an IgG4 antibody. In some embodiments, the antibody is a human IgG4 antibody.

### EXAMPLES

In order that the present disclosure may be more fully understood, the following examples are set forth. It should be understood that these examples are for illustrative purposes only and are not to be construed as limiting this disclosure in any manner.

### Example 1. Risk-Based Modeling Approach for Use of a 2× Diafiltration Buffer Concentrate

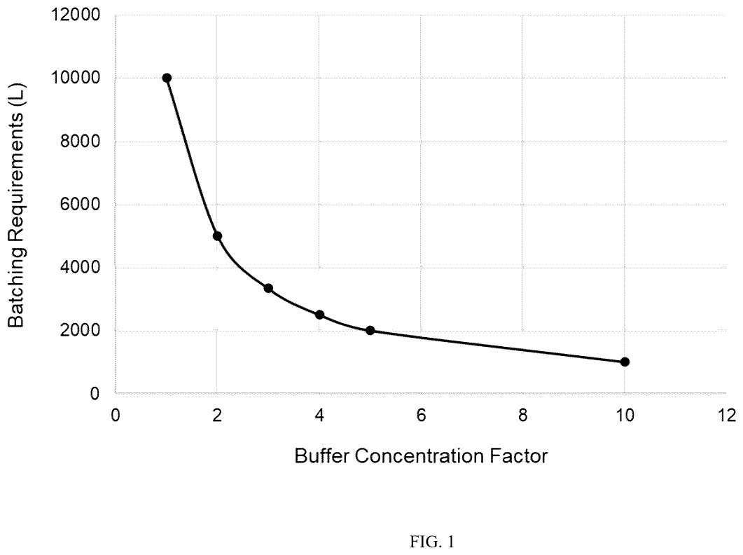

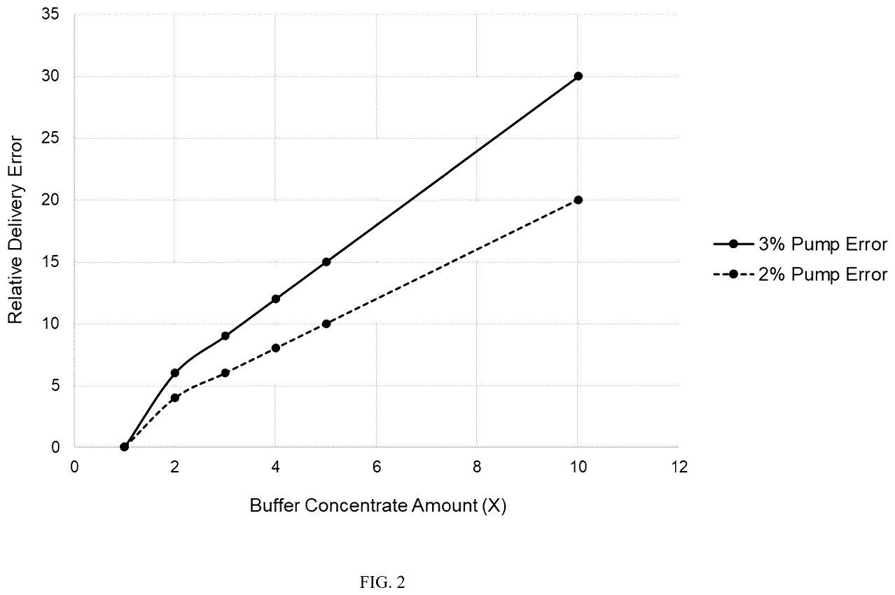

A risk-based modeling approach was used to assess the process impact of intensifying an ultrafiltration/diafiltration (UF/DF) unit operation for formulating a monoclonal antibody. Specifically, process modeling was used to assess the risk associated with employing diafiltration buffer concentrates during various phases of the UF/DF process by batching at a concentration above 1× of the target diafiltration concentration and diluting in-line with water during UF/DF operations to achieve the target 1× concentration. A higher concentration factor reduces buffer batching volume and associated resource usage (FIG. 1). However, overdilution or underdilution of a diafiltration buffer due to pump error (FIG. 2) during process steps such as diafiltration and product recovery post-diafiltration can impact critical quality attributes (CQAs, such as, e.g., protein concentration, osmolality, and/or pH), so the process modeling examined the impact of relative dilution accuracy of a 2× diafiltration buffer concentrate during different steps of the UF/DF process on product pool CQAs.

The risk-based modeling approach used in this study accounted for the Donnan effect, which is a phenomenon of differential partitioning of excipients due to the protein of interest being retained across the membrane and the thermodynamic activity of the permeate and retentate being equal. A first-principles-based model to predict the impact of the Donnan effect on UF/DF operations for various products and buffer types has been previously described (Ladwig et al., “Mechanistic model of pH and excipient concentration during ultrafiltration and diafiltration processes of therapeutic antibodies. Biotechnology Progress 2020 September; 36(5):e2993. doi: 10.1002/btpr.2993. Epub 2020 Apr. 7) and was employed here.

The model was first used to assess the relationship between the diafiltration buffer concentration of an excipient, final UF/DF pool concentration of the excipient, and the final UF/DF pool osmolality. While osmolality can be estimated from first principles based on predicted solute concentrations, experimental data were used in this model. Next, various scenarios for the accuracy of dilution of a 2× diafiltration buffer concentrate were simulated assuming a pump dilution accuracy of 90%, 95%, 105%, or 110% of target by modeling the diafiltration buffer concentration (model input) with the same level of variability. Batching variability for the diafiltration buffer was not accounted for.

The associated change in osmolarity for the final UF/DF pool was assessed for four conditions: (1) using the 2× diafiltration buffer concentrate with dilution for the entire process, including the recovery phase, (2) using the 2× diafiltration buffer concentrate with dilution for the entire process, excluding the recovery phase, (3) using the 2× diafiltration buffer concentrate with dilution for 6 of the 7 diavolumes and 1× buffer (no dilution) for the remainder (final 1 diavolume of DF and the recovery), and (4) using the 2× diafiltration buffer concentrate with dilution for 6.5 of the 7 diavolumes and 1× buffer (no dilution) for the remainder (final 0.5 diavolume of DF and the recovery). For each simulation, the predicted pool excipient concentration was used to predict the pool osmolality.

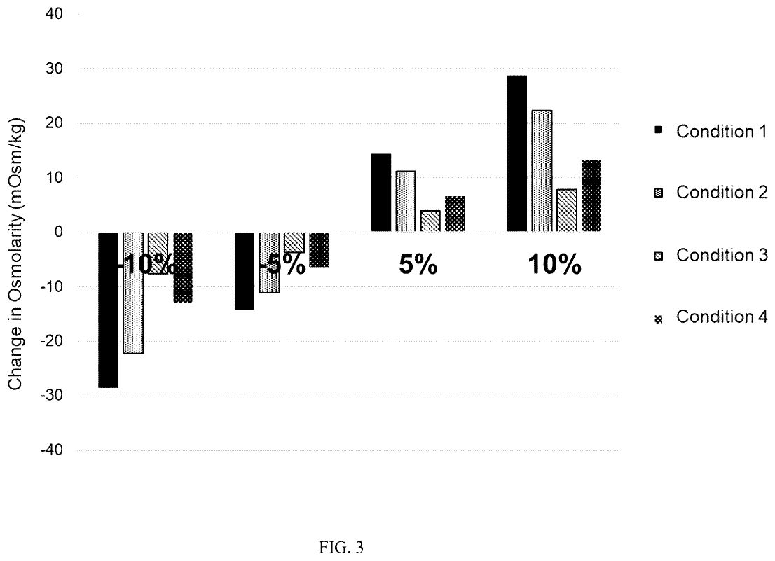

FIG. 3 shows the relative changes in predicted osmolality for the four UF/DF process conditions as compared to a traditional process in which a 1× buffer (corresponding to 100% dilution accuracy) is used for the entirety of the process from equilibration through recovery. As shown in FIG. 3, greater variability in the buffer dilution accuracy of ±10% leads to greater variability in the predicted osmolality. Additionally, restricting the use of the diafiltration buffer concentrate to the equilibration and diafiltration phases reduces the variability in predicted osmolality, and further restricting the use of the concentrated buffer to only a portion of diafiltration phase further reduces the variability.

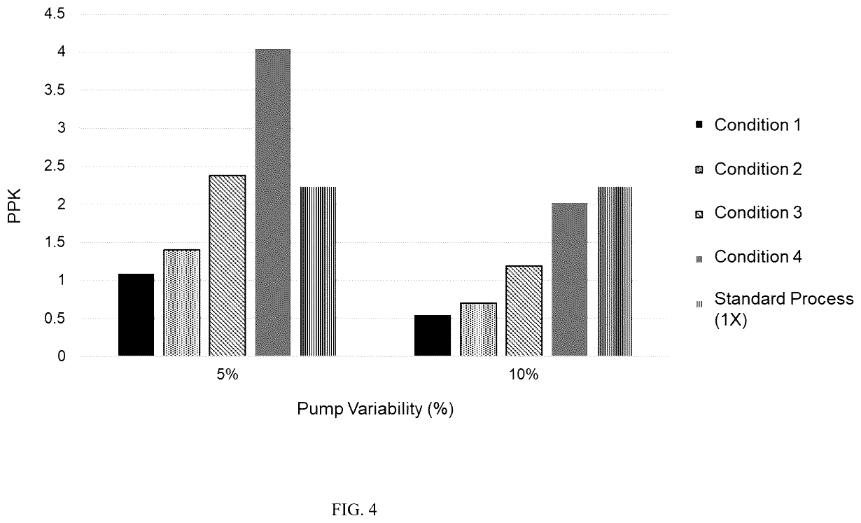

FIG. 4 shows the process capability (Ppk) relative to the product specification limits for osmolality for the simulated scenarios, assuming a worst-case uniform distribution for the pump variability.

The equation for calculating Ppk is shown in equation 1, where USL is the upper limit on the variable of interest, LSL is the lower limit on the variable of interest, x̅ is the average value of the variable of interest, and σ is the standard deviation. For this analysis, x̅ is the average drug substance osmolality measured experimentally, and σ is calculated using equation 2 which shows the standard deviation for a uniform distribution with maximum value y and minimum value x. A uniform distribution is a worst-case assumption for this type of analysis as it weights 5% error as equally likely with other values in the range (e.g., 1%, 2%, etc.). This may be realized in the event of systemic error in the pump output over the entirety of a process, but represents a worst case compared to a more likely operation in which the pump has periods of greater accuracy.

$$Ppk = min ( \frac{USL - \overline{x}}{3 σ} , \frac{\overline{x} - L S L}{3 σ} ) \qquad ( 1 )$$

$$σ = \sqrt{\frac{{( y - x )}^{2}}{12}} \qquad ( 2 )$$

The scenario that assumes ±5% pump error and a separate 1× buffer used for the recovery phase (Condition (2)) was determined to be a desirable operating strategy, as this level of accuracy is achievable on a large-scale UF/DF skid, the DF process does not include a change in dilution strategy (i.e., from 2× to 1× for the final diavolume), and the predicted osmolality exhibits a Ppk (>1.3). Osmolality is used as a performance indicator in this analysis as other parameters (i.e. pH), would be less sensitive to improper dilution for typical diafiltration buffers.

### Example 2. Risk-Based Modeling Approach for Assessing Ultrafiltration Overconcentration and Expedited Buffer Exchange During Diafiltration Using Buffer Concentrates

A risk-based modeling approach similar to that described in Example 1 was used to assess the process impact of intensifying an ultrafiltration/diafiltration (UF/DF) unit operation by using an elevated ultrafiltration concentration target with subsequent dilution with diafiltration buffer at the start of diafiltration. The intent of this approach is to reduce diafiltration volume requirements by removing more excipients during the ultrafiltration process and potentially increase the permeation rate in the early stages of diafiltration. The use of 2× diafiltration buffer concentrates at the beginning of the diafiltration process to expedite buffer exchange was also assessed.

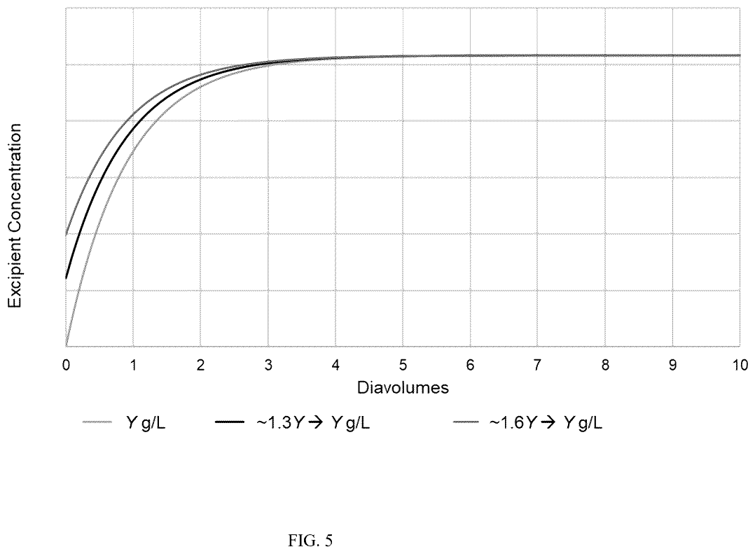

Specifically, simulations were conducted to assess the following 3 “overconcentration” scenarios: (1) concentrating to Y g/L, (2) concentrating to ˜1.3Y g/L, and (3) concentrating to ˜1.6Y g/L, where Y g/L is the intended concentration post-diafiltration. This initial overconcentration helps lower the overall excipient levels prior to diafiltration which results in a more efficient process. FIG. 5 shows the simulated concentration of an excipient under each of these conditions, where diafiltration buffer was used prior to the start of diafiltration to dilute the UF pool to a concentration of Y g/L.

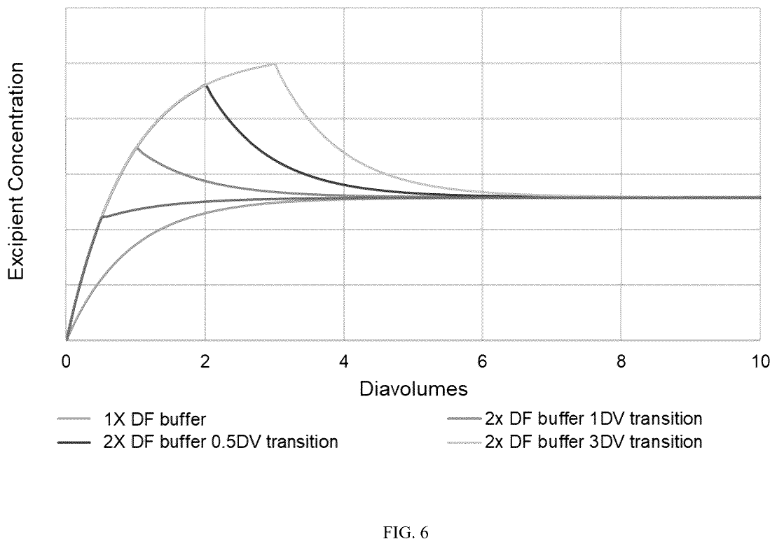

Additionally, the impact of expediting buffer exchange using a 2× diafiltration buffer concentrate for (1) 0.5 diavolumes (DV); (2) 1 diavolumes; (3) 2 diavolumes; and (4) 3 diavolumes of the diafiltration process before transitioning to use of a 1× diafiltration buffer was examined. FIG. 6 shows the simulated concentration of an excipient for each of these scenarios.

Next, various scenarios for dilution of the UF pool with DF buffer or expedited buffer exchange were simulated using a first-principles modeling approach previously described elsewhere (Ladwig et al., “Mechanistic model of pH and excipient concentration during ultrafiltration and diafiltration processes of therapeutic antibodies. Biotechnology Progress 2020 September; 36(5):e2993. doi: 10.1002/btpr.2993. Epub 2020 Apr. 7). For each simulation, the predicted UF/DF pool excipient concentration was used to predict the UF/DF pool osmolality, similar to the simulations of Example 1. Table 1 shows 6 scenarios compared at a fixed excipient target concentration, which was selected based on the equilibrium excipient concentration for a full buffer exchanged (i.e. ˜10 DVs). Note, 5% of target was used as a metric for measuring buffer exchange efficiency.

TABLE 1
Comparison of Conditions

| | 2X DF Buffer | Post-UF | DF | 5% of End of |
|---|---|---|---|---|
| | Transition | Concentration | Concentration | DF Excipient |
| Intensification Condition | Point (DV) | Target (g/L) | (g/L) | Concentration (DV) |
| Traditional Process (1X DF | N/A | Y | Y | 2.70 |
| Buffer; No Initial Dilution) | | | | |
| Overconcentration | N/A | ~1.3Y | Y | 2.45 |
| Overconcentration | N/A | ~1.6Y | Y | 2.25 |
| 2X DF Buffer Concentrate | 0.5 | Y | Y | 1.45 |
| 2X DF Buffer Concentrate | 1.0 | Y | Y | 2.71 |
| 2X DF Buffer Concentrate | 2.0 | Y | Y | 4.48 |
| 2X DF Buffer Concentrate | 3.0 | Y | Y | 5.66 |

### Example 3. Properties of Diluted 2× Buffer Concentrate

A study was performed to verify that target diafiltration (DF) buffer properties could be obtained using a 2× buffer concentrate recipe followed by 1:1 dilution with water.

In order to achieve a 1×DF buffer target composition of 272 mM proline, 10 mM acetate, pH 4.1, a 2× buffer concentrate recipe was created by doubling all buffer components (excipient-grade glacial acetic acid and L-proline) such that the resulting target composition was 544 mM proline, 20 mM acetate, pH 4.1. This 2× recipe was batched and tested, followed by 1:1 dilution with deionized water to achieve a 1× buffer, which was subsequently tested. Triplicate batches were prepared and tested. Testing of the buffers consisted of pH and conductivity measurements at 20-22° C. using an “Orion Star” “A Series” benchtop meter (Thermo Fisher Scientific), and osmolality measurements using an “OsmoTECH XT” Osmometer (Advanced Instruments).

Table 2 shows average results (with standard deviation in parentheses; N=3) for the diluted 2× buffer, as well as the target properties for the 1× buffer. All results fell within the desired ranges, demonstrating the feasibility and robustness of a strategy involving 1:1 dilution of a 2× concentrate recipe (based on 2× component amounts versus a neat buffer recipe), in terms of achieving target buffer properties.

TABLE 2
Diluted 2X Buffer Concentrate Properties

| | 2X DF Buffer Followed by | | 1X DF Buffer |
|---|---|---|---|
| | 1:1 Dilution with Water | | Targets |
| pH | 4.0 | (<0.1) | 4.0-4.2 |
| Conductivity (μS/cm) | 156 | (2) | 140-180 |
| Osmolality (mOsm/kg) | 302 | (2) | 261-319 |

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer to obtain a second composition,
wherein the first diafiltration buffer is prepared by diluting a diafiltration buffer concentrate.

2. The method of claim 1, wherein the diafiltering comprises diafiltering the first concentrated composition with 6 to 10 diavolumes of the first diafiltration buffer.

3. The method of claim 1 or claim 2, wherein the method further comprises:
optionally concentrating the second composition to obtain a second concentrated composition;
flushing the equilibrated UF/DF membrane filter with a second diafiltration buffer to obtain a recovery composition, wherein the second diafiltration buffer is not prepared by diluting a diafiltration buffer concentrate; and
combining the recovery composition and the second composition or the second concentrated composition to obtain a third composition.

4. The method of claim 3, wherein the first diafiltration buffer and the second diafiltration buffer have substantially the same composition.

5. The method of claim 3 or claim 4, wherein concentrating the second composition comprises ultrafiltering using the equilibrated UF/DF membrane filter.

6. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with the first diafiltration buffer and a second diafiltration buffer to obtain a second composition,
wherein:
the first diafiltration buffer is prepared by diluting a diafiltration buffer concentrate;
the second diafiltration buffer is not prepared by diluting a diafiltration buffer concentrate; and
the first diafiltration buffer and the second diafiltration buffer have substantially the same composition.

7. The method of claim 6, wherein the diafiltering comprises:
diafiltering with the first diafiltration buffer for 6.5 diavolumes followed by diafiltering with the second diafiltration buffer for 0.5 diavolumes; or
diafiltering with the first diafiltration buffer for 6 diavolumes followed by diafiltering with the second diafiltration buffer for 1 diavolume.

8. The method of claim 6 or claim 7, wherein the method further comprises:
optionally concentrating the second composition to obtain a second concentrated composition;
flushing the equilibrated UF/DF membrane filter with a third diafiltration buffer to obtain a recovery composition, wherein the third diafiltration buffer is not prepared by diluting a diafiltration buffer concentrate; and
combining the recovery composition and the second composition or the second concentrated composition to obtain a third composition.

9. The method of claim 8, wherein the first diafiltration buffer, the second diafiltration buffer, and the third diafiltration buffer have substantially the same composition.

10. The method of any one of claims 6 to 9, wherein concentrating the second composition comprises ultrafiltering using the equilibrated UF/DF membrane filter.

11. The method of any one of claims 1 to 10, wherein the first diafiltration buffer is prepared by diluting a diafiltration buffer concentrate with water.

12. The method of any one of claims 1 to 11, wherein the first diafiltration buffer is prepared by diluting a diafiltration buffer concentrate at an intended dilution ratio of 1:1.

13. The method of any one of claims 1 to 12, wherein the diluting is performed using an in-line dilution system.

14. The method of any one of claims 1 to 13, wherein the diluting is continuously performed using an in-line dilution system.

15. The method of claim 13 or claim 14, wherein a pump of the in-line dilution system has a relative dilution accuracy in the range of 90% to 110% of an intended dilution ratio.

16. A method for preparing a formulation comprising a recombinant protein, the method comprising:
equilibrating an ultrafiltration/diafiltration (UF/DF) membrane filter in a first diafiltration buffer to obtain an equilibrated UF/DF membrane filter;
concentrating a first composition comprising a recombinant protein by ultrafiltration using the equilibrated UF/DF membrane filter to obtain a first concentrated composition; and
diafiltering the first concentrated composition with a second diafiltration buffer and a third diafiltration buffer to obtain a second composition, wherein the second diafiltration buffer is a concentrate of the third diafiltration buffer.

17. The method of claim 16, wherein the second diafiltration buffer is at least a 2× concentrate of the third diafiltration buffer.

18. The method of claim 16 or claim 17, wherein the second diafiltration buffer is a 2× concentrate of the third diafiltration buffer.

19. The method of any one of claims 16 to 18, wherein the first diafiltration buffer and the third diafiltration buffer have substantially the same composition.

20. The method of any one of claims 16 to 19, wherein the diafiltering comprises:
diafiltering with the second diafiltration buffer for 0.5 diavolumes followed by diafiltering with the third diafiltration buffer for 6.5 to 9.5 diavolumes; or
diafiltering with the second diafiltration buffer for one diavolume followed by diafiltering with the third diafiltration buffer for 6 to 9 diavolumes; or
diafiltering with the second diafiltration buffer for two diavolumes followed by diafiltering with the third diafiltration buffer for 5 to 8 diavolumes; or
diafiltering with the second diafiltration buffer for three diavolumes followed by diafiltering with the third diafiltration buffer for 4 to 7 diavolumes.

21. The method of any one of claims 1 to 20, wherein the recombinant protein is present in the first concentrated composition at a concentration that is at least 100% of a concentration of the recombinant protein in the second composition and optionally diluted with a diafiltration buffer prior to the diafiltering, optionally wherein the first concentrated composition is diluted such that the concentration of the recombinant protein in the composition post-dilution is substantially the same as the concentration in the second composition.

22. The method of any one of claims 1 to 21, wherein the recombinant protein is present in the first concentrated composition at a concentration that is at least 120% of a concentration of the recombinant protein in the second composition and optionally diluted with a diafiltration buffer prior to the diafiltering, optionally wherein the first concentrated composition is diluted such that the concentration of the recombinant protein in the composition post-dilution is substantially the same as the concentration in the second composition.

23. A method for preparing a formulation comprising a recombinant protein, the method comprising:
concentrating a first composition comprising a recombinant protein by ultrafiltration to obtain a first concentrated composition; and
diafiltering the first concentrated composition with a first diafiltration buffer and a second diafiltration buffer to obtain a second composition, wherein:
the first diafiltration buffer is a concentrate of the second diafiltration buffer; and
the recombinant protein is present in the first concentrated composition at a concentration that is at least 120% of a concentration of the recombinant protein in the second composition.

24. The method of claim 23, wherein the first diafiltration buffer is at least a 2× concentrate of the second diafiltration buffer.

25. The method of claim 23 or claim 24, wherein the diafiltering comprises:
diafiltering with the first diafiltration buffer for 0.5 diavolumes followed by diafiltering with the second diafiltration buffer for 6.5 to 9.5 diavolumes; or
diafiltering with the first diafiltration buffer for one diavolume followed by diafiltering with the second diafiltration buffer for 6 to 9 diavolumes; or
diafiltering with the first diafiltration buffer for two diavolumes followed by diafiltering with the second diafiltration buffer for 5 to 8 diavolumes; or
diafiltering with the first diafiltration buffer for three diavolumes followed by diafiltering with the second diafiltration buffer for 4 to 7 diavolumes.
